(19) United States
(12) Patent Application Publication
(10) Pub. No.: US 2023/0339880 A1
(43) Pub. Date: Oct. 26, 2023

(54) COMPOSITIONS OF AJULEMIC ACID AND USES THEREOF

(71) Applicant: Corbus Pharmaceuticals, Inc., Norwood, MA (US)

(72) Inventors: David Richard BOOTH, Edinburgh (GB); Chaoyi DENG, Shanghai (CN); Robert Paul DISCORDIA, Denver, NC (US); Feng GUO, Shanghai (CN); Clifton David LEIGH, Somerville, MA (US); Kristos Adrian MOSHOS, Watertown, MA (US); Hayley Ann REECE, Dalkeith (GB); Abdolsamad TADAYON, Shanghai (CN); Tingting XU, Shanghai (CN); Hang ZHANG, Shanghai (CN)

(21) Appl. No.: 17/766,638

(22) PCT Filed: Oct. 9, 2020
(86) PCT No.: PCT/US2020/055152

§ 371 Date: Apr. 5, 2022

Related U.S. Application Data
(60) Provisional application No. 62/925,416, filed on Oct. 24, 2019.

(30) Foreign Application Priority Data
Oct. 11, 2019 (WO) PCT/CN2019/110575

(51) Int. Cl. C07D 311/80

(52) U.S. Cl. CPC C07D 311/80; C07B 2200/13

(57) ABSTRACT

The invention relates to crystalline forms of (6aR,10aR)-1-Hydroxy-6, 6-dimethyl-3-(2-methyl-2-octanyl)-6a,7,10,10a-tetrahydro-6H-benzo[c]chromene-9-carboxylic acid (ajulemic acid), including pharmaceutical compositions comprising a crystalline form of ajulemic acid and methods of making a crystalline form of ajulemic acid. The invention also relates to the use of pharmaceutical compositions comprising a crystalline form of ajulemic acid for the treatment of disease, including inflammatory diseases and fibrotic diseases.

### BACKGROUND OF THE INVENTION

Tetrahydrocannabinol (THC) is the major psychoactive constituent of marijuana. In addition to mood-altering effects, THC has been reported to exhibit other activities, some of which may have therapeutic value. The potential therapeutic value of THC has led to a search for related compounds which minimize the psychoactive effects, while retaining the activities of potential medicinal value.

One such related cannabinoid is (6aR,10aR)-1-hydroxy-6,6-dimethyl-3-(2-methyl-2-octanyl)-6a,7,10,10a-tetrahydro-6H-benzo[c]chromene-9-carboxylic acid (also known as ajulemic acid, AJA, JBT-101, resunab, anabasum, or lenabasum). Ajulemic acid has been investigated for its potential therapeutic benefits in a number of diseases, including fibrotic diseases and inflammatory diseases, for which there is a need for new therapies with improved safety and efficacy profiles.

Drugs currently used to treat chronic, serious diseases with chronic inflammation and fibrosis are divided broadly into several groups: non-steroidal anti-inflammatory drugs, anti-malarial agents, systemic corticosteroids, and immunosuppressive agents, each with its own disadvantages in certain subjects, depending upon the health of the subject being treated, the disease being treated, and the severity of the disease.

Treatment with ajulemic acid may offer a new therapeutic modality for diseases, including fibrotic diseases and inflammatory diseases. In particular, ajulemic acid may provide an improved efficacy and/or safety profile over available treatment options for such diseases.

The invention features compositions including crystalline forms of ajulemic acid, which may be used to improve the stability, shelf-life, pharmacokinetics, and/or dosing of ajulemic acid formulations. The invention also provides methods for making crystals of ajulemic acid and methods of using crystals of ajulemic acid for the treatment of disease, including inflammatory diseases and fibrotic diseases.

### SUMMARY OF THE INVENTION

The invention provides compositions and methods relating to crystalline forms of (6aR,10aR)-1-hydroxy-6,6-dimethyl-3-(2-methyl-2-octanyl)-6a,7,10,10a-tetrahydro-6H-benzo[c]chromene-9-carboxylic acid (ajulemic acid). The invention features crystals of ajulemic acid, pharmaceutical compositions including crystals of ajulemic acid, and methods of making crystals of ajulemic acid. The invention also features the use of pharmaceutical compositions including crystals of ajulemic acid for the treatment of diseases, including inflammatory diseases (e.g., scleroderma, systemic lupus erythematosus, or dermatomyositis) and fibrotic diseases (e.g., scleroderma or cystic fibrosis).

In a first aspect, the invention features crystals of ajulemic acid (e.g., a solid crystalline form of ajulemic acid) having at least one peak at diffraction angle 2θ at each of 7.1°±0.2°, 7.5°±0.2°, and 9.9±0.2° as measured by X-ray Powder Diffraction (XRPD). In some embodiments, the crystals of ajulemic acid have at least one peak at diffraction angle 2θ at each of 7.1°±0.2°, 7.5°±0.2°, and 9.9°±0.2°, and have one or more additional peaks at diffraction angle 2θ of 14.2°±0.2°, 16.1°±0.2°, 19.1°±0.2°, 19.3°±0.2°, 20.5°±0.2°, and/or 21.9°±0.2°, as measured by XRPD. In some embodiments, the crystals of ajulemic acid have at least one peak at diffraction angle 2θ at each of 7.1°±0.2°, 7.5°±0.2°, and 9.9°±0.2°, and 19.3±0.2°, as measured by XRPD. In some embodiments, the crystals of ajulemic acid have at least one peak at diffraction angle 2θ at each of 7.1°±0.2°, 7.5°±0.2°, and 9.9°±0.2°, and 21.9±0.2°, as measured by XRPD. In some embodiments, the crystals of ajulemic acid have at least one peak at diffraction angle 2θ at each of 7.1°±0.2°, 7.5°±0.2°, and 9.9°±0.2°, and 20.5±0.2°, as measured by XRPD. In some embodiments, the crystals of ajulemic acid have at least one peak at diffraction angle 2θ at each of 7.1°±0.2°, 7.5°±0.2°, and 9.9±0.2°, and 19.1±0.2°, as measured by XRPD. In some embodiments, the crystals of ajulemic acid have at least one peak at diffraction angle 2θ at each of 7.1°±0.2°, 7.5°±0.2°, and 9.9.2°±0.2°, and 16.1±0.2°, as measured by XRPD. In some embodiments, the crystals of ajulemic acid have at least one peak at diffraction angle 2θ at each of 7.1°±0.2°, 7.5°±0.2°, 9.9±0.2°, and 14.2°±0.2° as measured by XRPD.

In another aspect, the crystals of ajulemic acid have at least one peak at diffraction angle 2θ at each of 7.1°±0.2°, 7.5°±0.2°, and 14.2°±0.2°. In some embodiments, the crystals of ajulemic acid have at least one peak at diffraction angle 2θ at each of 7.1°±0.2°, 7.5°±0.2°, and 14.2°±0.2°, and have one or more additional peaks at diffraction angle 2θ of 9.9°±0.2°, 16.1°±0.2°, 19.1°±0.2°, 19.3°±0.2°, 20.5°±0.2°, and/or 21.9°±0.2°, as measured by XRPD. In some embodiments, the crystals of ajulemic acid have at least one peak at diffraction angle 2θ at each of 7.1°±0.2°, 7.5°±0.2°, and 14.2°±0.2°, and 19.3±0.2°, as measured by XRPD. In some embodiments, the crystals of ajulemic acid have at least one peak at diffraction angle 2θ at each of 7.1°±0.2°, 7.5°±0.2°, and 14.2°±0.2°, and 21.9±0.2°, as measured by XRPD. In some embodiments, the crystals of ajulemic acid have at least one peak at diffraction angle 2θ at each of 7.1°±0.2°, 7.5°±0.2°, and 14.2°±0.2°, and 20.5±0.2°, as measured by XRPD. In some embodiments, the crystals of ajulemic acid have at least one peak at diffraction angle 2θ at each of 7.1°±0.2°, 7.5°±0.2°, and 14.2°±0.2°, and 19.1±0.2°, as measured by XRPD. In some embodiments, the crystals of ajulemic acid have at least one peak at diffraction angle 2θ at each of 7.1°±0.2°, 7.5°±0.2°, and 14.2°±0.2°, and 16.1±0.2°, as measured by XRPD. In some embodiments, the crystals of ajulemic acid have at least one peak at diffraction angle 2θ at each of 7.1°±0.2°, 7.5°±0.2°, and 14.2°±0.2°, and 9.9±0.2°, as measured by XRPD.

In some embodiments, the crystals of ajulemic acid have three or more (e.g., three or more, four or more, five or more, six or more, seven or more, eight or more, nine or more, ten or more, eleven or more, twelve or more, thirteen or more, or fourteen or more) peaks listed in Table 1 as measured by XRPD. In some embodiments, the crystals of ajulemic acid have all of the peaks at the diffraction angles 2θ as measured by XRPD provided in Table 1. Table 1 shows all peaks with a relative intensity of greater than or equal to 10% and corresponds to the XRPD trace of Example 8 and FIG. 4. Each peak in Table 1 is considered to have an associated error of ±0.2°.

TABLE 1
XRPD of Crystal Form B of Ajulemic Acid

| 2θ (°) | Relative Intensity (%) |
|---|---|
| 7.09 | 100 |
| 7.47 | 82 |
| 9.53 | 26 |
| 9.85 | 33 |
| 10.12 | 25 |
| 13.40 | 21 |
| 14.00 | 14 |
| 14.22 | 56 |
| 14.56 | 13 |
| 14.70 | 21 |
| 14.96 | 14 |
| 16.09 | 33 |
| 17.05 | 54 |
| 17.37 | 23 |
| 17.93 | 23 |
| 18.06 | 21 |
| 18.39 | 17 |
| 19.08 | 34 |
| 19.27 | 80 |
| 19.50 | 19 |
| 19.78 | 11 |
| 20.38 | 31 |
| 20.46 | 45 |
| 21.19 | 23 |
| 21.38 | 13 |
| 21.63 | 12 |
| 21.87 | 48 |
| 22.31 | 19 |
| 22.51 | 11 |
| 23.28 | 23 |
| 24.05 | 18 |

In another aspect, the invention features crystals of ajulemic acid (e.g., a solid crystalline form of ajulemic acid) having at least one peak at each of 143.4 ppm±0.2 ppm, 150.6 ppm±0.2 ppm, and 153.8 ppm±0.2 ppm, as measured by ¹³C solid state Nuclear Magnetic Resonance (ssNMR). In some embodiments, the crystals have at least one peak at 175.5 ppm±0.2 ppm±0.2 ppm, as measured by ¹³C ssNMR.

In some embodiments, the crystals of ajulemic acid have three or more (e.g., three or more, four or more, five or more, six or more, seven or more, eight or more, nine or more, ten or more, eleven or more, twelve or more, thirteen or more, or fourteen or more) peaks listed in Table 2 as measured by ¹³C ssNMR. In some embodiments, the crystals of ajulemic acid have all of the peaks as measured by ¹³C ssNMR provided in Table 2. Table 2 shows corresponds to the ¹³C ssNMR characterization of crystals form B of ajulemic acid of Example 15 and FIG. 21. Each peak in Table 2 is considered to have an associated error of ±0.2 ppm.

TABLE 2
¹³C ssNMR of Crystal Form B of Ajulemic Acid
Peak v(F1) [ppm]

175.5
173.2
156.1
155.2
153.8
150.6
148.5
143.4
141.4
131.4
111.2
110.1
108.4
107.6
107.1
105.4
78.5
76.3
46.4
46.0
44.3
42.8
37.9
37.6
32.1
31.1
30.6
29.5
28.9
28.0
26.5
25.0
23.0
20.2
18.6
14.6
13.7

In another aspect, the invention features crystals of ajulemic acid having two or more peaks listed in Table 1 as measured by XRPD and two or more peaks listed in Table 2 as measured by ¹³C ssNMR. In some embodiments, the crystals of ajulemic acid have two peaks at diffraction angle 2θ selected from of 7.1°±0.2°, 7.5°±0.2°, and 14.2°±0.2°, as measured by XRPD, and the crystals of ajulemic acid have two peaks selected from 143.4 ppm±0.2 ppm, 150.6 ppm±0.2 ppm, and 153.8 ppm±0.2 ppm, as measured by ¹³C ssNMR.

In some embodiments, the crystals of ajulemic acid have at least one peak at diffraction angle 2θ at each of 7.1°±0.2° and 7.5°±0.2°, as measured by XRPD, and at least one peak at each of 143.4 ppm±0.2 ppm and 150.6 ppm±0.2 ppm, as measured by ¹³C ssNMR.

In some embodiments, the crystals of ajulemic acid have at least one peak at diffraction angle 2θ at each of 7.1°±0.2° and 14.2°±0.2°, as measured by XRPD, and at least one peak at each of 143.4 ppm±0.2 ppm and 150.6 ppm±0.2 ppm, as measured by ¹³C ssNMR.

In some embodiments, the crystals of ajulemic acid have at least one peak at diffraction angle 2θ at each of 7.5°±0.2° and 14.2°±0.2°, as measured by XRPD, and at least one peak at each of 143.4 ppm±0.2 ppm and 150.6 ppm±0.2 ppm, as measured by ³C ssNMR.

In another aspect, the invention features a pharmaceutical composition including crystals of ajulemic acid (e.g., crystals of ajulemic acid as described herein) and a pharmaceutically acceptable excipient.

In some embodiments, the pharmaceutical composition including the crystals of ajulemic acid is a tablet (e.g., a tablet including crystals of ajulemic acid and a pharmaceutically acceptable excipient). In some embodiments, the tablet is prepared by compressing the crystals of ajulemic acid and one or more polymers. In some embodiments, the tablet includes a lubricating agent, a semi-permeable coating, a rate-controlling polymer, or a binding agent (e.g., hydroxyalkyl cellulose, hydroxyalkylalkyl cellulose, hydroxypropyl methyl cellulose, or polyvinylpyrrolidone).

In some embodiments, the pharmaceutical composition including the crystals of ajulemic acid is a capsule. In some embodiments, the capsule includes an excipient (e.g., lactose, glucose, sucrose, mannitol, corn starch, potato starch, or cellulose). In some embodiments, the capsule is formulated for sustained release. In some embodiments, the capsule is a hard gel capsule or a soft gel capsule.

In another aspect, the invention features a pharmaceutical composition including ajulemic acid, wherein the pharmaceutical composition is prepared by dissolving crystals of ajulemic acid (e.g., crystals of ajulemic acid as described herein) into a suitable pharmaceutical excipient (e.g., a pharmaceutical vehicle, such as a liquid, gel, or cream vehicle).

In another aspect, the invention features a method of making a pharmaceutical composition including ajulemic acid, wherein the pharmaceutical composition is prepared by dissolving crystals of ajulemic acid (e.g., crystals of ajulemic acid as described herein) into a suitable pharmaceutical excipient (e.g., a pharmaceutical vehicle, such as a liquid, gel, or cream vehicle).

In some embodiments, the pharmaceutical excipient is selected from water, a saline solution, an oil (e.g., petroleum oil, an animal oil, an oil of synthetic origin, a mineral oil, or a vegetable oil such as peanut oil, soybean oil, or sesame oil), glycerol, an aqueous dextrose solution, propylene glycol, or ethanol.

In some embodiments, the pharmaceutical composition is a capsule (e.g., a liquid capsule or a gel capsule), a liquid (e.g., a liquid formulated for parenteral administration, such as intravenous administration, for oral administration, or for ophthalmic administration), an ointment, cream, or gel (e.g., an ointment, cream, or gel, formulated for ophthalmic administration or topical administration), a patch, or an inhaled formulation.

In some embodiments, the pharmaceutical composition including ajulemic acid is a unit dose in the form of a tablet (e.g., a pressed tablet). In some embodiments, the unit dose includes 5±1 mg, 7±2 mg, 10±2 mg, 15±3 mg, 20±4 mg, 25±4 mg, 30±5 mg, 35±5 mg, or 40±8 mg of ajulemic acid. In some embodiments the tablet is administered once daily (e.g., 5±1 mg administered once daily, 7±2 mg administered once daily, 10±2 mg administered once daily, 15±3 mg administered once daily, 20±4 mg administered once daily, 25±4 mg administered once daily, 30±5 mg administered once daily, 35±5 mg administered once daily, or 40±8 mg administered once daily). In some embodiments the tablet is administered twice daily (e.g., 5±1 mg administered twice daily, 7±2 mg administered twice daily, 10±2 mg administered twice daily, 15±3 mg administered twice daily, 20±4 mg administered twice daily, 25±4 mg administered twice daily, 30±5 mg administered twice daily, 35±5 mg administered twice daily, or 40±8 mg administered twice daily).

In some embodiments, the pharmaceutical composition including ajulemic acid is a unit dose in the form of a capsule (e.g., a gel capsule or a liquid capsule). In some embodiments, the unit dose includes 5±1 mg, 7±2 mg, 10±2 mg, 15±3 mg, 20±4 mg, 25±4 mg, 30±5 mg, 35±5 mg, or 40±8 mg of ajulemic acid. In some embodiments the capsule is administered once daily (e.g., 5±1 mg administered once daily, 7±2 mg administered once daily, 10±2 mg administered once daily, 15±3 mg administered once daily, 20±4 mg administered once daily, 25±4 mg administered once daily, 30±5 mg administered once daily, 35±5 mg administered once daily, or 40±8 mg administered once daily). In some embodiments the capsule is administered twice daily (e.g., 5±1 mg administered twice daily, 7±2 mg administered twice daily, 10±2 mg administered twice daily, 15±3 mg administered twice daily, 20±4 mg administered twice daily, 25±4 mg administered twice daily, 30±5 mg administered twice daily, 35±5 mg administered twice daily, or 40±8 mg administered twice daily).

In some embodiments, the pharmaceutical composition including ajulemic acid is in a unit dosage form including from 1 to 100 mg of ajulemic acid (e.g., from 1 mg to 2 mg, 2 mg to 5 mg, 4 mg to 10 mg, 6 mg to 15 mg, 8 mg to 20 mg, 10 mg to 25 mg, 12 mg to 30 mg, 20 mg to 35 mg, 25 mg to 40 mg, or 30 mg to 40 mg, from 40 mg to 100 mg ajulemic acid). For example, each unit dosage form can contain 3±1 mg, 4±1 mg, 5±1 mg, 8±2 mg, 10±2 mg, 12±3 mg, 15±3 mg, 20±4 mg, 22±4 mg, 27±4 mg, 30±5 mg, 35±5 mg, or 40±8 mg ajulemic acid.

In some embodiments, the pharmaceutical composition including ajulemic acid is administered once daily, twice daily, or three times daily.

In another aspect, the invention features a method of treating a subject having an inflammatory disease, where the method includes administering to the subject a pharmaceutical composition including crystals of ajulemic acid and a pharmaceutically acceptable excipient (e.g., any of the pharmaceutical compositions described herein, such as a pharmaceutical composition including crystals of ajulemic acid or a pharmaceutical composition prepared by dissolving crystals of ajulemic acid into a suitable pharmaceutical excipient) in an amount sufficient to treat the inflammatory disease.

In some embodiments, the inflammatory disease is scleroderma (e.g., systemic sclerosis, localized scleroderma, or sine scleroderma), systemic lupus erythematosus, dermatomyositis, acquired immune deficiency syndrome (AIDS), multiple sclerosis, rheumatoid arthritis, psoriasis, diabetes (e.g., Type 1 diabetes), cancer, asthma, atopic dermatitis, an autoimmune thyroid disorder, ulcerative colitis, Crohn's disease, stroke, ischemia, a neurodegenerative disease (e.g., Alzheimer's disease or Parkinson's disease), amyotrophic lateral sclerosis (ALS), chronic traumatic encephalopathy (CTE), chronic inflammatory demyelinating polyneuropathy, an autoimmune inner ear disease, uveitis, iritis, or peritonitis.

In another aspect, the invention features a method of treating a subject having a fibrotic disease, the method including administering to the subject a pharmaceutical composition including crystals of ajulemic acid and a pharmaceutically acceptable excipient (e.g., any of the pharmaceutical compositions described herein, such as a pharmaceutical composition including crystals of ajulemic acid or a pharmaceutical composition prepared by dissolving crystals of ajulemic acid into a suitable pharmaceutical excipient) in an amount sufficient to treat the fibrotic disease.

In some embodiments, the fibrotic disease is scleroderma (e.g., systemic sclerosis, localized scleroderma, or sine scleroderma), liver cirrhosis, interstitial pulmonary fibrosis, idiopathic pulmonary fibrosis, Dupuytren's contracture, keloids, cystic fibrosis, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, or organ fibrosis (e.g., dermal fibrosis, lung fibrosis, liver fibrosis, kidney fibrosis, or heart fibrosis).

In some embodiments of any of the aspects or embodiments described herein, the crystals of ajulemic acid have a melting point of 168° C.±5° C., 169° C.±5° C., 170° C.±5° C., 171° C.±5° C., 172° C.±5° C., or 173° C.±5° C. Most preferably, the crystals of ajulemic acid have a melting point of 170° C.±5° C. (e.g., 170° C.±4° C., 170° C.±3° C., 170° C.±2° C., or 170° C.±1° C.). In a preferred embodiment, the crystals of ajulemic acid have at least one peak at diffraction angle 2θ at each of 7.1°±0.2°, 7.5°±0.2°, and 14.2°±0.2° as measured by XRPD and a melting point of 170° C.±5° C.

In some embodiments of any of the aspects or embodiments described herein, the crystals of ajulemic acid have an endothermic onset at 168° C.±5° C., 169° C.±5° C., 170° C.±5° C., 171° C.±5° C., 172° C.±5° C., or 173° C.±5° C. in their differential scanning calorimetry (DSC) profile. Preferably, the crystals have an endothermic onset at 170° C.±5° C. (e.g., 170° C.±4° C., 170° C.±3° C., 170° C.±2° C., or 170° C.±1° C.) in their differential scanning calorimetry (DSC) profile.

In some embodiments of any of the aspects or embodiments described herein, the crystals of ajulemic acid have an endothermic peak at 170° C.±5° C., 171° C.±5° C., 172° C.±5° C., 173° C.±5° C., 174° C.±5° C., or 175° C.±5° C. in their differential scanning calorimetry (DSC) profile. Preferably the crystals have an endothermic peak at 172° C.±5° C. (e.g., 172° C.±4° C., 172° C.±3° C., 172° C.±2° C., or 172° C.±1° C.) in their differential scanning calorimetry (DSC) profile.

In some embodiments of any of the aspects or embodiments described herein, the crystals of ajulemic acid have a unit cell of the space group P2₁2₁2₁, having dimensions of a=13.8951 Å, b=14.5553 Å, and c=22.0051 Å and α=90°, β=90°, γ=90° as determined by X-ray diffractometry and/or a unit cell volume of 4450 Å³.

In another aspect, the invention features a method of producing crystals of ajulemic acid (e.g., any of the crystals of ajulemic acid described herein) wherein ajulemic acid is dissolved in and subsequently isolated from (e.g., re-crystallized) in heptanes (e.g., n-heptane), dichloromethane, pentane, hexane, chloroform, dichloroethane, cyclohexane, water, isomers of alkane, or a suitable mixture thereof. Preferably, the ajulemic acid is dissolved in and subsequently isolated from (e.g., re-crystallized) in heptanes (e.g., n-heptane), dichloromethane, water, or cyclohexane.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “treat” or “treatment” includes administration of a compound, e.g., by any route, e.g., orally, topically, parenterally, ophthalmically, or by inhalation to a subject. The compound can be administered alone or in combination with one or more additional compounds. Treatments may be sequential, with the present compound being administered before or after the administration of other agents. Alternatively, compounds may be administered concurrently. The subject, e.g., a patient, can be one having a disorder (e.g., a disorder as described herein), a symptom of a disorder, or a predisposition toward a disorder. Treatment is not limited to curing or complete healing, but can result in one or more of alleviating, relieving, altering, partially remedying, ameliorating, improving or affecting the disorder, reducing one or more symptoms of the disorder or the predisposition toward the disorder. In an embodiment the treatment (at least partially) alleviates or relieves symptoms related to a fibrotic disease. In an embodiment the treatment (at least partially) alleviates or relieves symptoms related to an inflammatory disease. In one embodiment, the treatment reduces at least one symptom of the disorder or delays onset of at least one symptom of the disorder. The effect is beyond what is seen in the absence of treatment.

The term “pharmaceutical composition” refers to the combination of an active agent with an excipient (e.g., a diluent, carrier, or vehicle), inert or active, making the composition especially suitable for diagnostic or therapeutic use in vivo or ex vivo.

Aa used herein, the term “pharmaceutically acceptable excipient” refers to an inactive substance that serves as the vehicle, diluent, or carrier for an active substance. A pharmaceutically acceptable excipient is one that after administered to or upon a subject, does not cause undesirable physiological effects. The excipient in the pharmaceutical composition must be “acceptable” also in the sense that it is compatible with the active ingredient. One or more solubilizing agents can be utilized as pharmaceutical excipients for delivery of an active compound. Examples of pharmaceutically acceptable excipients include, but are not limited to, vehicles, adjuvants, additives, polymers, and diluents to achieve a composition usable as a dosage form. Examples of excipients are provided throughout the disclosure and include, for example, magnesium stearate, cellulose, sodium lauryl sulfate, starch, glucose, lactose, sucrose, mannitol, gelatin, sodium stearate, glycerol monostearate, talc, and sodium chloride. Pharmaceutical excipients can be liquids, such as water and oils, including those of petroleum, animal, vegetable or synthetic origin, such as peanut oil, soybean oil, mineral oil, sesame oil and the like. Pharmaceutical excipients can include saline, gum acacia, gelatin, starch paste, talc, keratin, urea, and the like. In addition, auxiliary, stabilizing, thickening, lubricating and coloring agents can be used. Water can be the pharmaceutical excipient when the active compound is administered intravenously. Saline solutions and aqueous dextrose and glycerol solutions can also be employed as liquid excipients, particularly for injectable solutions. Suitable pharmaceutical excipients also include glycerol, propylene glycol, water, and ethanol. The present compositions, if desired, can also contain minor amounts of wetting or emulsifying agents, or pH buffering agents.

### BRIEF DESCRIPTION OF THE FIGURES

FIG. 1 is a Differential Scanning Calorimetry (DSC) trace of crystal form A of ajulemic acid. An endothermic event is observed with an onset of about 91° C. and a peak of about 98° C.

FIG. 2 is a Differential Scanning Calorimetry (DSC) trace of crystal form B of ajulemic acid. An endothermic event is observed with an onset of about 170° C. and a peak of approximately 172° C.

FIG. 3. is an X-Ray Powder Diffraction (XRPD) trace of crystal form A of ajulemic acid. The corresponding diffraction angles 2θ (°) for crystal form A are provided in Table 5.

FIG. 4 is an X-Ray Powder Diffraction (XRPD) trace of crystal form B of ajulemic acid. The corresponding diffraction angles 2θ (°) for crystal form B are provided in Table 1.

FIG. 5 is a comparison of the simulated and experimental XRPD results for crystal form B.

FIG. 6 is a series of Variable Temperature X-Ray Powder Diffraction (VT-XRPD) traces of crystal form B of ajulemic acid. VT-XRPD was performed as described in Example 8. VT-XRPD indicated that the endothermic observed in DSC at approximately 170° C. is melt or decomposition of crystal form B.

FIG. 7 is a Thermogravimetric Analysis/Dynamic Temperature Analysis (TGA/DTA) of crystal form A. TGA indicates a 0.7% wt. loss from the onset of about 210° C. DTA indicates an endothermic thermal event with onset at about 94° C.

FIG. 8 is a Thermogravimetric Analysis/Dynamic Temperature Analysis (TGA/DTA) of crystal form B. TGA indicates a 0.9% wt. loss from the onset to about 210° C. DTA indicates an endothermic event with an onset at about 169° C.

FIG. 9 is a Dynamic Vapor Sorption (DVS) isotherm analysis of crystal form B.

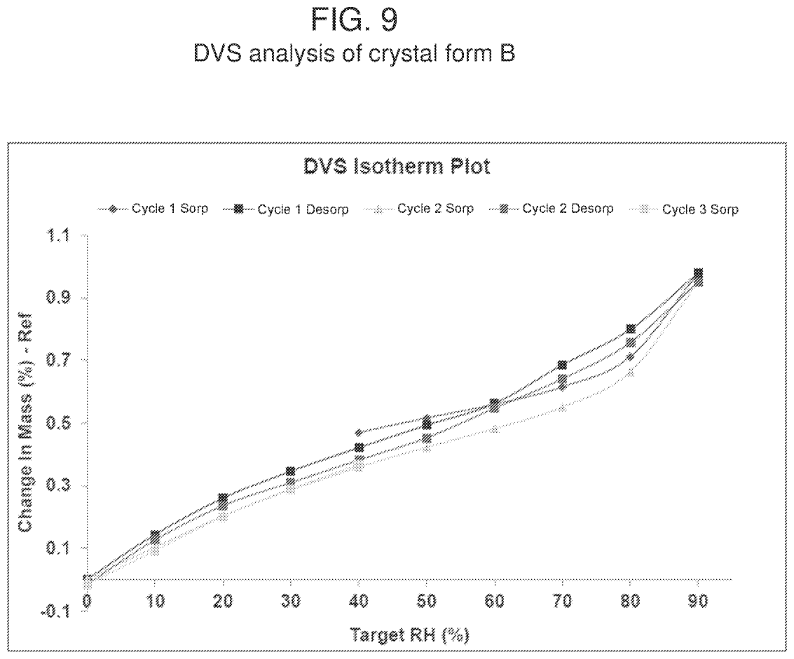

FIG. 10 is a DVS kinetic analysis of crystal form B.

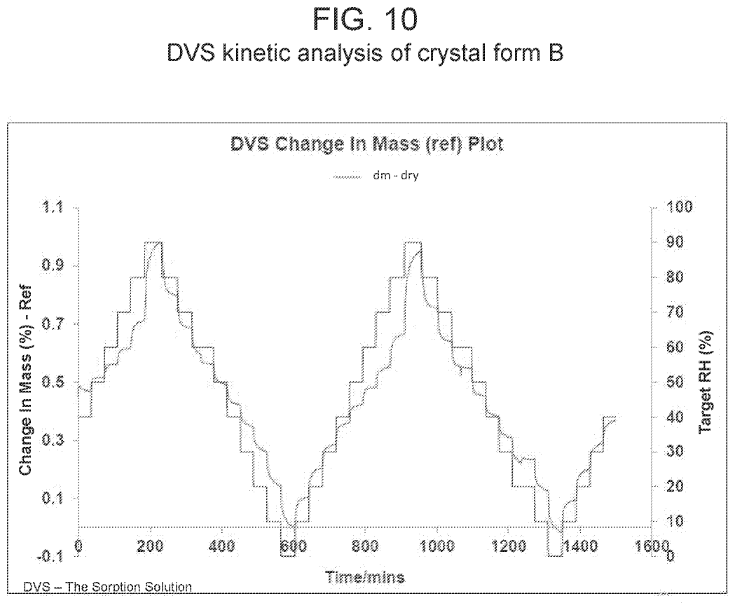

FIG. 11 is a proton Nuclear Magnetic Resonance (¹H-NMR) spectrum of crystal form B of ajulemic acid.

FIG. 12 is a Heteronuclear Single Quantum Coherence Nuclear Magnetic Resonance (HSQC-NMR) spectrum of crystal form B of ajulemic acid.

FIG. 13 is an image depicting the asymmetric unit of crystal form B of ajulemic acid as determined by single crystal X-ray diffraction analysis. The asymmetric unit contains two complete molecules of ajulemic acid.

FIG. 14 is an image depicting the crystal packing of a unit cell of crystal form B of ajulemic acid as viewed from unit cell axis a.

FIG. 15 is an image depicting the crystal packing of a unit cell of crystal form B of ajulemic acid as viewed from unit cell axis b.

FIG. 16 is an image depicting the crystal packing of a unit cell of crystal form B of ajulemic acid as viewed from unit cell axis c.

FIG. 17 is an image depicting crystal forms A and B after a 1-month open air stability test demonstrating the greater stability of crystal form B. After the 1-month test crystal form A has become an orange-brown solid, while crystal form B has maintained a white appearance demonstrating the greater air stability of crystal form B. This study was performed as described in Example 14, which includes further HPLC characterization of impurities in crystals form A and crystal form B.

FIG. 18 is a High-Performance Liquid Chromatography (HPLC) chromatogram of crystal form A after the 1-month open air stability test.

FIG. 19 is a HPLC chromatogram of crystal form B after the 1-month open air stability test.

FIG. 20 is a ¹³C solid state Nuclear Magnetic Resonance (ssNMR) spectrum of crystal form A. The corresponding peaks are provided in Table 12.

FIG. 21 is a ¹³C ssNMR spectrum of crystal form B. The corresponding peaks are provided in Table 2.

FIG. 22 is a ¹³C ssNMR spectrum of amorphous ajulemic acid. The corresponding peaks are provided in Table 13.

FIG. 23 is a comparison of the ¹³C ssNMR spectra of crystal form A, crystal form B, and amorphous ajulemic acid.

FIG. 24 is an overlay of the ¹³C ssNMR spectra from about 110 ppm to about 210 ppm of crystal form A and crystal form B.

FIG. 25 is an overlay of the ¹³C ssNMR spectra from about 55 ppm to about 125 ppm of crystal form A and crystal form B.

FIG. 26 is an overlay of the ¹³C ssNMR spectra from about 0 ppm to about 60 ppm of crystal form A and crystal form B.

### DETAILED DESCRIPTION OF THE INVENTION

The invention features a crystalline polymorph of (6aR,10aR)-1-Hydroxy-6,6-dimethyl-3-(2-methyl-2-octanyl)-6a,7,10,10a-tetrahydro-6H-benzo[c]chromene-9-carboxylic acid (ajulemic acid) with improved physical properties, including stability. The crystalline polymorph of ajulemic acid described herein may be used to improve the stability, shelf-life, pharmacokinetics, and/or dosing of ajulemic acid formulations. The invention features crystals of ajulemic acid, pharmaceutical compositions including crystals of ajulemic acid, methods of making crystals of ajulemic acid, and the use of the pharmaceutical compositions for the treatment of diseases, including inflammatory diseases and fibrotic diseases.

### Ajulemic Acid

(6aR,10aR)-1-Hydroxy-6,6-dimethyl-3-(2-methyl-2-octanyl)-6a,7,10,10a-tetrahydro-6H-benzo[c]chromene-9-carboxylic acid (ajulemic acid) is a cannabinoid that is structurally related to THC, but which lacks the undesirable psychotropic effects associated with THC. As a result, ajulemic acid has been investigated for its potential therapeutic utility in a number of diseases including fibrotic diseases and inflammatory diseases.

Ajulemic acid has the following structure:

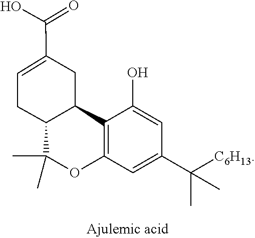

Ajulemic acid (e.g., a crystal form of ajulemic acid) may be an ultrapure formulation of ajulemic acid (e.g., lenabasum) including more than 95%, 96%, 97%, 98%, 99%, or 99.5% ajulemic acid and less than 5%, 4%, 3%, 2%, 1%, 0.5%, or 0.1% highly-active CB-1 impurities, e.g., HU-210. Ajulemic acid may be synthesized as described in U.S. Patent Publication No. 2015/0141501, which is incorporated herein by reference.

### Crystal Form B of Ajulemic Acid

Ajulemic acid may be subject to oxidative degradation, including oxidative degradation by air to produce a quinone derivative. As such, there is a need for preparations of ajulemic acid with greater stability towards oxidative degradation. There is also a need to improve the dosing (e.g., frequency or amount) of ajulemic acid in order to optimize the compliance, safety, and/or efficacy of a therapeutic regimen for the treatment of disease.

The invention provides compositions of crystalline ajulemic acid having a specific crystal form, “crystal form B,” which may increase the thermostability of ajulemic acid (e.g., increased stability towards oxidative degradation).

Crystal form B of ajulemic acid has been characterized, for example, by Differential Scanning Calorimetry (DSC) (see, e.g., Example 7), X-Ray Powder Diffraction (XRPD) (see, e.g., Example 8), Thermogravimetric Analysis/Dynamic Temperature Analysis (TGA/DTA) (see, e.g., Example 9), Dynamic Vapor Sorption (DVS) (see, e.g., Example 10), Nuclear Magnetic Resonance (NMR) (see, e.g., Example 11), single crystal X-ray diffraction analysis (SCXRD) (see, e.g., Example 12), thermodynamic solubility (see, e.g., Example 13), open air stability (see, e.g., Example 14), and solid state Nuclear Magnetic Resonance (ssNMR) (see, e.g., Example 15).

Crystal form B of ajulemic acid can be produced by crystallization or re-crystallization of ajulemic acid in a suitable solvent (e.g., heptane, dichloromethane, water, or cyclohexane). In some embodiments, the crystal form B of ajulemic acid has a residual level of solvent (e.g., heptane, dichloromethane, water, or cyclohexane) of about 0-50 ppm, about 50-100 ppm, about 100-200 ppm, about 200-500 ppm, about 500-1000 ppm, about 1000-1500 ppm, about 1500-2000 ppm, about 2000-2500 ppm, about 2500-5000 ppm, or about 5000-10000 ppm. The invention also contemplates crystallization or re-crystallization of ajulemic acid in all suitable solvents and solvent mixtures to produce crystal form B of ajulemic acid having the XRPD, DSC, and NMR characteristics described herein.

The thermostability and other characteristics (e.g., pharmacokinetics, dosing, or shelf-life) of crystal form B may be contrasted with prior crystal form A. Crystal form A is produced and characterized as described herein. New crystal form B is more thermodynamically stable, more oxidatively stable, and is less susceptible to gain and loss of water (e.g., equilibrating with ambient humidity levels) as compared to the previously observed crystal form A.

### Methods for Processing Crystals of Ajulemic Acid

The crystals of ajulemic acid described herein can include ajulemic acid particles having an effective particle size from about 1 micron to about 500 microns (e.g., about 1 micron to about 10 microns, about 10 microns to about 100 microns, about 100 microns to about 200 microns, about 200 microns to about 300 microns, about 300 microns to about 400 microns, or about 400 microns to about 500 microns). In some embodiments, the crystals of ajulemic acid described herein can include ajulemic acid particles having an effective particle size of less than about 1 micron (e.g., nanoparticulate formulations). In the preferred embodiments, the starting ajulemic acid composition is predominantly crystalline, most preferably crystal form B of ajulemic acid.

The crystals of ajulemic acid may be micronized. Micronized crystalline particles of ajulemic acid can be made by using any method known in the art for achieving the desired particle sizes. Useful methods include, for example, milling, homogenization, supercritical fluid fracture, or precipitation techniques. Exemplary methods are described in U.S. Pat. Nos. 4,540,602; 5,145,684; 5,518,187; 5,718,388; 5,862,999; 5,665,331; 5,662,883; 5,560,932; 5,543,133; 5,534,270; 5,510,118; and 5,470,583, each of which is specifically incorporated by reference.

Milling to Obtain Crystals of Ajulemic Acid

In one approach, the crystals of ajulemic acid are milled in order to obtain micron or submicron particles. The milling process can be a dry process, e.g., a dry roller milling process, a jet milling process, or a wet process, i.e., wet-grinding. A wet-grinding process is described in U.S. Pat. Nos. 4,540,602; 5,145,684; and 6,976,647, the disclosures of which are hereby incorporated by reference. Thus, the wet-grinding process can be practiced in conjunction with a liquid dispersion medium and a dispersing or wetting agent such as described in these publications. Useful liquid dispersion media include safflower oil, ethanol, n-butanol, hexane, or propylene glycol, among other liquids selected from known organic pharmaceutical excipients (see U.S. Pat. Nos. 4,540,602 and 5,145,684), and can be present in an amount of 2.0-70%, 3-50%, or 5-25% by weight based on the total weight of the ajulemic acid, in the formulation.

Homogenization to Obtain Crystals of Ajulemic Acid

Ajulemic acid particles can also be prepared by high pressure homogenization (see, e.g., U.S. Pat. No. 5,510,118). In this approach ajulemic acid particles are dispersed in a liquid dispersion medium and subjected to repeated homogenization to reduce the particle size of the ajulemic acid particles to the desired effective average particle size. The ajulemic acid particles can be reduced in size in the presence of at least one or more dispersing agents or wetting agents. Alternatively, the ajulemic acid particles can be contacted with one or more dispersing agents or wetting agents either before or after attrition. Other materials, such as a diluent, can be added to the ajulemic acid/dispersing agent mixture before, during, or after the size reduction process. For example, unprocessed ajulemic acid can be added to a liquid medium in which it is essentially insoluble to form a premix (e.g., about 0.1-60% w/w ajulemic acid and about 20-60% w/w dispersing agents or wetting agents). The apparent viscosity of the premix suspension is preferably less than about 1000 centipoise. The premix can then be transferred to a microfluidizer and circulated continuously, first at low pressures, and then at maximum capacity (e.g., 3,000 to 30,000 psi) until the desired particle size reduction is achieved.

Milling with Simethicone

Foaming during the micronizing process can present formulation issues and can have negative consequences for particle size reduction. For example, high levels of foam or air bubbles in the mill can cause a drastic increase in viscosity, rendering the milling process inoperable. Even a very low level of air presence can dramatically reduce milling efficiency, rendering the desired particle size unachievable. This may be due to the resultant air in the mill cushioning the milling balls and limiting grinding efficiency. The air can also form a microemulsion with the milled ingredients, which presents many issues with respect to the delivery of an accurate dose and palatability. Addition of a small amount of simethicone is a very effective anti-foaming technique which minimizes milling variability or the requirement for special handling techniques to avoid the introduction of air into the milling process.

The Use of Wetting and Dispersing Agents

The ajulemic acid particles can be prepared with the use of one or more wetting and/or dispersing agents, which are, e.g., adsorbed on the surface of the ajulemic acid particle. The ajulemic acid particles can be contacted with wetting and/or dispersing agents either before, during or after size reduction. Generally, wetting and/or dispersing agents fall into two categories: non-ionic agents and ionic agents. The most common non-ionic agents are excipients which are contained in classes known as binders, fillers, surfactants and wetting agents. Limited examples of non-ionic surface stabilizers are hydroxypropylmethylcellulose, polyvinylpyrrolidone, Plasdone, polyvinyl alcohol, Pluronics, Tweens and polyethylene glycols (PEGs). Ionic agents are typically organic molecules bearing an ionic bond such that the molecule is charged in the formulation, such as long chain sulfonic acid salts.

Excipients, such as wetting and dispersing agents, can be applied to the surface of the ajulemic acid particulate via spray drying, spray granulation, or a spray layering process. These procedures are well known to those skilled in the art. It is also common to add additional excipients prior to removal of solvent from the particulate suspension to aid in the dispersion of the solid composition in the medium in which the solid composition will be exposed (e.g. saliva) to further prevent agglomeration and/or particle size growth of the small ajulemic acid particles. An example of such an additional excipient is a redispersing agent. Suitable redispersing agents include, without limitation, sugars, polyethylene glycols, urea and quaternary ammonium salts.

### Pharmaceutical Compositions

As described above, the pharmaceutical compositions of the invention additionally include a pharmaceutically acceptable excipient, which, as used herein, includes any and all solvents, diluents, vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Some examples of materials which can serve as pharmaceutically acceptable excipients include, but are not limited to, sugars such as lactose, glucose, mannitol, and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil, safflower oil, sesame oil, olive oil, corn oil, and soybean oil; glycols, such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, distearoyl phosphatidylethanolamine (DSPE) and its pegylated esters, such as DSPE-PEG750 and DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred. Buffering agents such as magnesium hydroxide and aluminum hydroxide; alginic acid; pyrogen-free water; isotonic saline; Ringer's solution; ethyl alcohol; and phosphate buffer solutions; as well as non-toxic compatible lubricants such as sodium lauryl sulfate and magnesium stearate; as well as coloring agents, releasing agents, coating agents, sweetening, flavoring and perfuming agents, preservatives and antioxidants can also be present in the composition, according to the judgment of the formulator.

Pharmaceutically acceptable carriers, adjuvants and vehicles that may be used in the pharmaceutical compositions of this invention also include, but are not limited to, ion exchangers; alumina; aluminum stearate; lecithin; self-emulsifying drug delivery systems (SEDDS); self-microemulsifying drug delivery systems (SMEDDS), such as d-E-tocopherol polyethylene-glycol 1000 succinate; surfactants used in pharmaceutical compositions such as Tweens or other similar polymeric delivery matrices; serum proteins such as human serum albumin; buffer substances such as phosphates; glycine; sorbic acid; potassium sorbate; partial glyceride mixtures of saturated vegetable fatty acids; water; salts, electrolytes, such as protamine sulfate, sodium hydrogen phosphate, potassium hydrogen phosphate, sodium chloride, zinc salts, or magnesium trisilicate; polyvinyl pyrrolidone; cellulose-based substances; polyethylene glycol; sodium carboxymethylcellulose; polyacrylates; waxes; polyethylene-polyoxypropylene-block polymers; polyethylene glycol; and wool fat. Cyclodextrins such as alpha-, beta-, and gamma-cyclodextrin, or chemically modified cyclodextrin derivatives such as hydroxyalkylcyclodextrins, including 2- and 3-hydroxypropyl-beta cyclodextrins, or other solubilized derivatives may also be advantageously used to enhance delivery of compounds of the formulae described herein that can be used in the methods of the invention for preventing and/or treating fibrotic conditions.

In certain embodiments, unit dosage formulations are compounded for immediate release, though unit dosage formulations compounded for delayed or prolonged release of one or both agents are also disclosed.

Viscosity modifiers that may be used in pharmaceutical compositions of the present invention include, but are not limited to, caprylic/capric triglyceride (Migliol 810), isopropyl myristate (IPM), ethyl oleate, triethyl citrate, dimethyl phthalate, benzyl benzoate, and various grades of polyethylene oxide. High viscosity liquid carriers used in sustained release pharmaceutical compositions include, but are not limited to, sucrose acetate isobutyrate (SAIB) and cellulose acetate butyrate (CAB 381-20).

Non-limiting examples of binding agents that may be used in pharmaceutical compositions of the present invention include but are not limited to a hydroxyalkyl cellulose, a hydroxyalkylalkyl cellulose, hydroxypropyl methyl cellulose, or a polyvinylpyrrolidone.

Non-limiting examples of osmotic agents that may be used in pharmaceutical compositions of the present invention include, but are not limited to, sorbitol, mannitol, sodium chloride, or other salts. Non-limiting examples of biocompatible polymers employed in the contemplated pharmaceutical compositions include, but are not limited to, poly(hydroxy acids), polyanhydrides, polyorthoesters, polyamides, polycarbonates, polyalkylenes, polyalkylene glycols, polyalkylene oxides, polyalkylene terepthalates, polyvinyl ethers, polyvinyl esters, polyvinyl halides, polyvinylpyrrolidone, polysiloxanes, poly(vinyl alcohols), poly(vinyl acetate), polystyrene, polyurethanes and co-polymers thereof, synthetic celluloses, polyacrylic acids, poly(3-hydroxybutyric acid), poly(3-hydroxyvaleric acid), poly(lactide-co-caprolactone), ethylene vinyl acetate, copolymers and blends thereof.

Non-limiting examples of hygroscopic polymers that may be employed in the contemplated pharmaceutical compositions include, but are not limited to, polyethylene oxide (e.g., Polyox®), cellulose, hydroxymethylcellulose, hydroxyethylcellulose, crosslinked polyacrylic acids, and xanthan gum.

Non-limiting examples of rate-controlling polymers the may be employed in the contemplated pharmaceutical compositions include, but are not limited to, polymeric acrylate, methacrylate lacquer or mixtures thereof, polymeric acrylate lacquer, methacrylate lacquer, an acrylic resin including a copolymer of acrylic and methacrylic acid esters, or an ammonium methacrylate lacquer with a plasticizer.

The above-described compositions, in any of the forms described herein, can be used for treating disease (e.g., fibrotic disease, inflammatory disease, or any other disease or condition described herein). An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered by any suitable route, e.g., parenterally, orally, nasally, rectally, topically, buccally, by ophthalmic administration, or by inhalation. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, an aqueous mannitol solution, water, Ringer's solution, and isotonic sodium chloride solution. In addition, fixed oils are conventionally employed as a solvent or suspending medium (e.g., synthetic mono- or diglycerides). Fatty acids, such as, but not limited to, oleic acid and its glyceride derivatives, are useful in the preparation of injectables, as are natural pharmaceutically acceptable oils, such as, but not limited to, olive oil or castor oil, or polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other pharmaceutical compositions can also be used for the purpose of formulation.

A composition for oral administration can be any orally acceptable dosage form including capsules, tablets, emulsions and aqueous suspensions, dispersions, and solutions. In some embodiments, the dosage form is an oral dosage form such as a pressed tablet, hard or soft gel capsule, enteric coated tablet, osmotic release capsule, or unique combination of excipients. In the case of tablets, commonly used excipients include, but are not limited to, lactose, mannitol, and corn starch. Lubricating agents, such as, but not limited to, magnesium stearate, also are typically added. For oral administration in a capsule form, useful diluents include, but are not limited to, lactose, mannitol, glucose, sucrose, corn starch, potato starch, or cellulose. In additional embodiments, the dosage form includes a capsule wherein the capsule contains a mixture of materials to provide a desired sustained release formulation. When aqueous suspensions or emulsions are administered orally, the active ingredient can be suspended or dissolved in an oily phase combined with emulsifying or suspending agents. If desired, certain sweetening, flavoring, or coloring agents can be added.

The pharmaceutical compositions can include a tablet coated with a semipermeable coating. In certain embodiments, the tablet includes two layers, a layer containing ajulemic acid (e.g. ultrapure ajulemic acid) and a second layer referred to as a “push” layer. The semi-permeable coating is used to allow a fluid (e.g., water) to enter the tablet and erode a layer or layers. In certain embodiments, this sustained release dosage form further includes a laser-drilled hole in the center of the coated tablet. The ajulemic acid-containing layer may include ajulemic acid, a disintegrant, a viscosity modifier, a binding agent, and/or an osmotic agent. The push layer includes a disintegrant, a binding agent, an osmotic agent, and/or a viscosity modifier. Non-limiting examples of materials that make up preferred semi-permeable layers include, but are not limited to cellulosic polymers such as cellulose acetate, cellulose acylate, cellulose diacylate, cellulose triacylate, cellulose diacetate, cellulose triacetate or any mixtures thereof; ethylene vinyl acetate copolymers, polyethylene, copolymers of ethylene, polyolefins including ethylene oxide copolymers (e.g., Engage® Dupont Dow Elastomers), polyamides, cellulosic materials, polyurethanes, polyether blocked amides, and copolymers (e.g., PEBAX®, cellulosic acetate butyrate and polyvinyl acetate). Non-limiting examples of disintegrants that may be employed in the above sustained release pharmaceutical compositions include, but are not limited to, croscarmellose sodium, crospovidone, sodium alginate, or similar excipients.

In further embodiments, the dosage form includes a tablet including a biocompatible matrix and ajulemic acid. The dosage form may also include a hard-shell capsule containing bio-polymer microspheres that contain the therapeutically-active agent. The biocompatible matrix and bio-polymer microspheres each contain pores for drug release and delivery. Each biocompatible matrix or bio-polymer microsphere is made up of a biocompatible polymer or mixture of biocompatible polymers. The matrix or microspheres can be formed by dissolving the biocompatible polymer and active agent (compound described herein) in a solvent and adding a pore-forming agent (e.g., a volatile salt). Evaporation of the solvent and pore forming agent provides a matrix or microsphere containing the active compound.

In additional embodiments, the dosage form includes a tablet, wherein the tablet contains ajulemic acid and one or more polymers and wherein the tablet can be prepared by compressing the ajulemic acid and one or more polymers. In some embodiments, the one or more polymers may include a hygroscopic polymer formulated with ajulemic acid. Upon exposure to moisture, the tablet dissolves and swells. This swelling allows the sustained release dosage form to remain in the upper GI tract. The swelling rate of the polymer mixture can be varied using different grades of polyethylene oxide.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients. In some preferred embodiments, the topical formulations include a material that would enhance absorption or penetration of the active agent(s) through the skin or other affected areas.

A topical composition contains a safe and effective amount of a dermatologically-acceptable excipient suitable for application to the skin. A “cosmetically-acceptable” or “dermatologically-acceptable” composition or component refers to a composition or component that is suitable for use in contact with human skin without undue toxicity, incompatibility, instability, or allergic response. The excipient enables an active agent and optional component to be delivered to the skin at an appropriate concentration(s). The excipient thus can act as a diluent, dispersant, solvent, or the like to ensure that the active materials are applied to and distributed evenly over the selected target at an appropriate concentration. The excipient can be solid, semi-solid, or liquid. The excipient can be in the form of a lotion, a cream, or a gel, in particular one that has a sufficient thickness or yield point to prevent the active materials from sedimenting. The excipient can be inert or possess dermatological benefits. It should also be physically and chemically compatible with the active components described herein, and should not unduly impair stability, efficacy, or other use benefits associated with the composition.

The present compositions may be formulated for sustained release (e.g., over a 6-hour period, over a 12-hour period, over a 24-hour period, or over a 48-hour period). In some embodiments, the sustained release dosage form includes a tablet or a capsule including particle cores coated with a suspension of active agent and a binding agent, and which are subsequently coated with a polymer. The polymer may be a rate-controlling polymer. In general, the delivery rate of the rate-controlling polymer is determined by the rate at which the active agent is dissolved.

In another embodiment, the composition is formulated to provide extended release. In some embodiments, the agent is formulated with an enteric coating. In an alternative embodiment, the agent is formulated using a biphasic controlled release delivery system, thereby providing prolonged gastric residence. For example, in some embodiments, the delivery system includes (1) an inner solid particulate phase formed of substantially uniform granules containing an active agent, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophilic polymers, one or more hydrophobic polymers and/or one or more hydrophobic materials such as one or more waxes, fatty alcohols and/or fatty acid esters, which may be compressed into tablets or filled into capsules. In some embodiments, the agent is incorporated into polymeric matrices composed of hydrophilic polymers that swell upon imbibition of water to a size that is large enough to promote retention of the dosage form in the stomach during the fed mode.

The ajulemic acid in the formulation may be formulated as a combination of fast-acting and controlled release forms.

The present compositions may be taken just prior to, or with, each of three meals, each of two meals, or one meal. In other embodiments, a composition disclosed herein can be administered one or more times daily (e.g., once daily, twice daily, or three times daily).

The pharmaceutical composition can be administered alone or in combination with one or more additional compounds. Treatments may be sequential, with the present compound being administered before or after the administration of other agents. Alternatively, compounds may be administered concurrently. Exemplary additional agents include an analgesic agent such as an opiate, an anti-inflammatory agent, or a natural agent such as a triglyceride-containing unsaturated fatty acid or isolated pure fatty acids such as eicosapentaenoic acid (EPA), dihomogamma linolenic acid (DGLA), docosahexaenoic acid (DHA) and others. In some embodiments, the therapeutic agents that can be used in the present methods are formulated in a single unit dose such that the agents are released from the dosage at different times.

### Methods of Treatment

In some embodiments of the invention, any of the above-described compositions, including any of the above-described pharmaceutical compositions, may be administered to a subject (e.g., a mammal, such as a human, cat, dog, horse, cow, goat, sheep, or pig) having a disease (e.g., a fibrotic disease or an inflammatory disease) in order to treat, prevent, or ameliorate the disease.

Inflammation

A therapeutically effective amount of any of the compositions described herein (e.g. a pharmaceutical composition comprising ajulemic acid, such as crystals of ajulemic acid) may be used to treat or prevent inflammatory disease.

Inflammatory diseases include, for example, scleroderma (e.g., systemic sclerosis, localized scleroderma, or sine scleroderma), systemic lupus erythematosus, dermatomyositis, acquired immune deficiency syndrome (AIDS), multiple sclerosis, rheumatoid arthritis, psoriasis, diabetes (e.g., Type 1 diabetes), cancer, asthma, atopic dermatitis, an autoimmune thyroid disorder, ulcerative colitis, Crohn's disease, stroke, ischemia, a neurodegenerative disease (e.g., Alzheimer's disease or Parkinson's disease), amyotrophic lateral sclerosis (ALS), chronic traumatic encephalopathy (CTE), chronic inflammatory demyelinating polyneuropathy, an autoimmune inner ear disease, uveitis, iritis, and peritonitis.

In some embodiments, inflammation can be assayed by measuring the chemotaxis and activation state of inflammatory cells. In some embodiments, inflammation can be measured by examining the production of specific inflammatory mediators such as interleukins, cytokines and eicosanoid mediators. In some embodiments, in vivo inflammation is measured by swelling and edema of a localized tissue or migration of leukocytes. Inflammation may also be measured by organ function such as in the lung or kidneys and by the production of pro-inflammatory factors. Inflammation may also be assessed by other suitable methods, including the improvement, amelioration, or slowing of the progression of one or more symptoms associated with the particular inflammatory disorder being treated. Other methods known to one skilled in the art may also be suitable methods for the assessment of inflammation and may be used to evaluate or score the response of the subject to treatment with ajulemic acid.

Fibrotic Diseases

A therapeutically effective amount of any of the compositions described herein (e.g. a pharmaceutical composition comprising ajulemic acid, such as crystals of ajulemic acid) may be used to treat or prevent fibrotic disease.

Fibrotic diseases include, for example, scleroderma (e.g., systemic sclerosis, localized scleroderma, or sine scleroderma), liver cirrhosis, interstitial pulmonary fibrosis, idiopathic pulmonary fibrosis, Dupuytren's contracture, keloids, cystic fibrosis, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, or organ fibrosis (e.g., dermal fibrosis, lung fibrosis, liver fibrosis, kidney fibrosis, or heart fibrosis).

Non-limiting examples of fibrosis include liver fibrosis, lung fibrosis (e.g., silicosis, asbestosis or idiopathic pulmonary fibrosis), oral fibrosis, endomyocardial fibrosis, retroperitoneal fibrosis, deltoid fibrosis, kidney fibrosis (including diabetic nephropathy), cystic fibrosis, and glomerulosclerosis. Liver fibrosis, for example, occurs as a part of the wound-healing response to chronic liver injury. Fibrosis can occur as a complication of haemochromatosis, Wilson's disease, alcoholism, schistosomiasis, viral hepatitis, bile duct obstruction, exposure to toxins, and metabolic disorders. Endomyocardial fibrosis is an idiopathic disorder that is characterized by the development of restrictive cardiomyopathy. In endomyocardial fibrosis, the underlying process produces patchy fibrosis of the endocardial surface of the heart, leading to reduced compliance and, ultimately, restrictive physiology as the endomyocardial surface becomes more generally involved. Oral submucous fibrosis is a chronic, debilitating disease of the oral cavity characterized by inflammation and progressive fibrosis of the submucosal tissues (lamina propria and deeper connective tissues). The buccal mucosa is the most commonly involved site, but any part of the oral cavity can be involved, even the pharynx. Retroperitoneal fibrosis is characterized by the development of extensive fibrosis throughout the retroperitoneum, typically centered over the anterior surface of the fourth and fifth lumbar vertebrae.

Treatment of fibrosis may be assessed by suitable methods known to one of skill in the art including the improvement, amelioration, or slowing of the progression of one or more symptoms associated with the particular fibrotic disease being treated.

Scleroderma

Scleroderma is a disease of the connective tissue characterized by inflammation and fibrosis of the skin and internal organs. Scleroderma has a spectrum of manifestations and a variety of therapeutic implications. It includes localized scleroderma, systemic sclerosis, scleroderma-like disorders, and sine scleroderma. Systemic sclerosis can be diffuse or limited. Limited systemic sclerosis is also called CREST (calcinosis, Raynaud's esophageal dysfunction, sclerodactyly, telangiectasia). Systemic sclerosis includes: scleroderma lung disease, scleroderma renal crisis, cardiac manifestations, muscular weakness including fatigue or limited CREST, gastrointestinal dysmotility and spasm, and abnormalities in the central, peripheral and autonomic nervous system.

The major symptoms or manifestations of scleroderma, and in particular of systemic sclerosis, are inappropriate excessive collagen synthesis and deposition, endothelial dysfunction, vasospasm, and collapse and obliteration of vessels by fibrosis. In terms of diagnosis, an important clinical parameter may be skin thickening proximal to the metacarpophalangeal joints. Raynaud's phenomenon may be a component of scleroderma. Raynaud's may be diagnosed by color changes of the skin upon cold exposure. Ischemia and skin thickening may also be symptoms of Raynaud's disease.

A therapeutically effective amount of any of the compositions described herein may be used to treat or prevent fibrosis. Fibrosis may be assessed by suitable methods known to one of skill in the art.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### General Methods

Synthesis of Ajulemic Acid

Ajulemic acid may be synthesized as known in the art. Preferably, ajulemic acid is an ultrapure formulation of ajulemic acid including more than 99% ajulemic acid and less than 1% (e.g., less than 0.5%, 0.1%, or 0.05%) highly-active CB-1 impurities, e.g., HU-210. Ajulemic acid may be synthesized as described in U.S. Patent Publication No. 2015/0141501, which is incorporated herein by reference.

High Performance Liquid Chromatography (HPLC) Analysis

HPLC analysis was conducted using a Waters Xbridge Shield RP18 4.6 mm×150 mm column (3.5 μm, PN 186003045). Detection was set to 230 nm and column temperature to 35° C., with a 1.0 mL/min flow rate and a 10 μL injection volume. The gradient program is displayed in Table 3.

TABLE 3
Gradient program for the HPLC analysis of crystal forms A and B.

| Mobile Phase A | 10 mM ammonium formate in Water (pH = 3.0) | | |
|---|---|---|---|
| Mobile Phase B | ACN/MeOH = 70/30, (v/v) | | |
| Gradient program | Time (min) | A % | B % |
| | Initial | 38 | 62 |
| | 30.00 | 25 | 75 |
| | 38.00 | 5 | 95 |
| | 48.00 | 5 | 95 |
| | 49.00 | 38 | 62 |
| | 56.00 | | stop |

### Example 1. Preparation of Crystal Form A of Ajulemic Acid

Preparation of Crystal Form A

A 3 kg batch of ajulemic acid was made according to standard protocols for the preparation of ajulemic acid (see, e.g., U.S. Patent Publication No. 2015/0141501), with 500 g of the immediate precursor to ajulemic acid being removed during synthesis as described in Example 2. The typical crystallization procedure was followed to isolate a previously known crystal form of ajulemic acid, crystal form A.
(1) The ajulemic acid was dissolved in acetonitrile (8.5-10 volumes total, telescoped from a solvent exchange) and was heated to 70-75° C. and held there for 0.5-2 hours, confirming that all solids were dissolved.
(2) The solution was cooled to 60-70° C. over 1-3 hours, then seeded with about 5 wt % of crystal form A of ajulemic acid.
(3) The seeded batch was held at 63-67° C. for 1-3 hours, then cooled to 2-7° C. over 8-12 hours and held at that temperature for another 5-12 hours before filtering.
(4) After filtration, the wet cake was dried under reduced pressure (≤0.08 MPa) at 20-30° C. for 6-12 hours before heating the vacuum oven to 50-55° C. Drying continued at this temperature until the acetonitrile level was ≤250 ppm.

The resulting batch of ajulemic acid took 11 days to dry. This batch was characterized by DSC and XRPD and identified as crystal form A.

### Example 2. Initial Observation of Crystal Form B

A novel and distinct crystal form, crystal form B, was obtained and identified. A 500 g portion of the immediate precursor to ajulemic acid was removed from the 3 kg batch described in Example 1. The 500 g portion was carried through to the synthesis of ajulemic acid and the ajulemic acid was isolated and crystallized by the standard procedure described in Example 1. The 500 g portion took 20 days to dry, significantly longer than the typical 7-15 days previously observed. Two endothermic events were observed in DSC analysis, one with an onset of 91.3° C. corresponding to crystal form A and a second smaller event with an onset of approximately 170° C., suggesting that the resulting ajulemic acid was a mixture of the known crystal form A and a new crystal form B.

50 g of ajulemic acid produced in this manner (e.g., ajulemic acid having both crystal forms A and B) was combined with an additional 100 g of ajulemic acid purified from the mother liquor of the batch described in Example 1 (crystal form not known). The resulting 150 g of ajulemic acid was dissolved in CH₂Cl₂ and the solution was concentrated to apparent dryness to produce a crystalline material having approximately 1900 ppm CH₂Cl₂. This crystalline material was characterized by DSC, XRPD, and NMR, which confirmed the presence of new crystal form B.

### Example 3. Small Scale Solvent Selection in Cyclohexane and n-Heptane

Potential solvents for crystal form B preparation were identified. Form B was the preferred form in water, cyclohexane, and heptane (see, e.g., Example 6). Water was eliminated from consideration for scale-up preparation of crystal form B due to poor mixing. An approximate solubility assessment was carried out on cyclohexane and heptane to select the most appropriate solvent using the following procedure:

Approximately 10 mg of crystal form A was weighed into a 1.5 mL screw-cap vial. 50 μL aliquots of solvent were added while stirring at 25° C. At 200 μL of solvent, the mixture in cyclohexane turned into an oil, while the n-heptane experiment continued to be a thin white slurry. The solids from the n-heptane experiment were isolated by centrifugation after about 2 hours stirring at 25° C. and analyzed by XRPD. The material in the cyclohexane experiment was observed to have recrystallized after about 2-5 hours stirring at 25° C. The cyclohexane mixture was left to stir for about 72 hours before the solids were isolated by centrifugation and analyzed by XRPD.

### Example 4. Preparation of Crystal Form B

Form B was prepared by slurry conversion in heptane at 25° C. using the following initial procedure:

Approximately 15 g of crystal form A was transferred to a 300 mL jacketed vessel. n-Heptane (75 mL, 200 mg/mL of crystal form A) was added in 5 equal portions at 25° C. and the mixture stirred at 120 rpm for 0.75 hours. The mixture was seeded with 25 mg of crystal form B (dried isolated material from the competitive slurry experiment above). Stirring at 150 rpm continued at 25° C. for about 64 hours. The slurry was then sampled, with solids from the sample isolated by centrifugation and a portion of them analyzed by XRPD. The remaining sampled solids were dried under vacuum at ambient temperature for about 1 hour. Stirring of the remaining slurry continued at 25° C. for a further 8 hours. The slurry was sampled after 4 hours and 8 hours, with the solids in each sample isolated by centrifugation and a portion of them analyzed by XRPD. The remaining sampled solids were dried under vacuum at ambient temperature for about 1-3 hours.

Another 75 mL of n-heptane was added to the remaining slurry (to achieve a 100 mg/mL slurry) and the mixture stirred at 200 rpm, still at 25° C. The slurry was sampled after about 15 hours, with the solids from the sample isolated by centrifugation and a portion of them analyzed by XRPD. The remaining sampled solids were dried under vacuum at ambient temperature for about 3 hours.

Another further 150 mL of n-heptane was added to the remaining slurry (to achieve a 50 mg/mL slurry) and the mixture stirred at 200 rpm, still at 25° C. The slurry was sampled after about 3 hours, with the solids from the sample isolated by centrifugation and a portion of them analyzed by XRPD. The remaining sampled solids were dried under vacuum at ambient temperature for about 3 hours.

The remaining slurry was cooled to 5° C. at 0.1° C./min. After about 20 minutes at 5° C., the solids were isolated by vacuum filtration using a 100 mm Büchner funnel and grade 1 filter paper. The filter cake was dried under vacuum at ambient temperature for about 17 hours.

The dried isolated solids were then re-slurried in heptane using the following procedure:

The dried isolated solids were transferred to a 300 mL jacketed vessel and rinsed in with 300 mL of n-heptane. The slurry was stirred at 240 rpm for about 16.5 hours at 25° C. The slurry was then sampled, with the solids from the sample isolated by centrifugation and a portion of them analyzed by XRPD. The remaining sampled solids were dried under vacuum at ambient temperature for about 1.5 hours.

After another 7.5 hours stirring at 25° C., the solids in the remaining slurry were isolated by vacuum filtration using a 100 mm Büchner funnel and grade 1 filter paper. The isolated solids were dried under vacuum at ambient temperature for about 14 hours.

### Example 5. Preparation of Amorphous Ajulemic Acid

Amorphous JBT-101 was prepared on about a 1 g scale by the following procedure:

Approximately 1.5 g of crystal form A was weighed into a 20 mL scintillation vial. Dichloromethane (DCM, 7.5 mL, making a 200 mg/mL concentration) was added and fully dissolved the crystal form A at ambient temperature. The solvent was removed by fast rotary evaporation producing a partially gum-like solid, which was sampled for XRPD. The material was re-dissolved in 10 mL (150 mg/mL concentration) DCM at ambient temperature and transferred to a 25 mL round-bottom flask. The solvent was again removed by fast rotary evaporation producing a partially gum-like solid. The material was redissolved in 12.5 mL of DCM and the solvent was again removed by fast rotary evaporation.

### Example 6. Competitive Slurries Procedure

Approximately 50 mg each of crystal form A and crystal form B were combined into a 1.5 mL screw cap vial with 0.5-1 mL of solvent or solvent system. If necessary, more solvent or solvent system was added to achieve a mobile slurry. The slurry was stirred for 48 hours at the indicated temperature (either 20° C. or 50° C.). The resulting material was isolated by centrifugation and analyzed by XRPD. The XRPD plates were then dried under vacuum at ambient temperature for 87 hours and XRPD analysis was repeated on the dried solids. The results of the competitive slurry analysis are provided in Table 4.

XRPD analysis showed that the recovered form was solvent dependent with no apparent dependence on temperature. Form A was recovered from recrystallization in acetone, acetonitrile, or ethyl acetate:heptane 50:50 v/v, and subsequent desolvation. Form B was recovered as an asolvate from recrystallization in heptane or dichloromethane. Drying of samples had no significant effect on the crystal form recovered.

TABLE 4
Competitive Slurries

| | XRPD of wet solids | | XRPD of dry solids | |
|---|---|---|---|---|
| Solvent system | 20 ° C. | 50 ° C. | 20 ° C. | 50 ° C. |
| Acetone | A | A | A | A |
| Acetonitrile | A | A | A | A |
| Heptane | B | B | B | B |
| Ethyl Acetate:Heptane | A* | A | A | A |
| 50:50 v/v | | | | |
| Dichloromethane | B | N/A | B | N/A |

*extra peaks observed

### Example 7. Differential Scanning Calorimetry (DSC) of Crystal Forms A and B

Differential Scanning Calorimetry (DSC)

Approximately 1-5 mg of material was weighed into an aluminum DSC pan and sealed non-hermetically with an aluminum lid. The sample pan was then loaded into a TA Instruments Discovery DSC 2500 differential scanning calorimeter equipped with a RC90 cooler. The sample and reference were heated to 240° C. at a scan rate of 10° C./min and the resulting heat flow response monitored. The sample was re-cooled to 20° C. and then reheated again to 240° C., all at 10° C./min. Nitrogen was used as the cell purge gas, at a flow rate of 50 cm³/min.

DSC Characterization of Crystal Form A

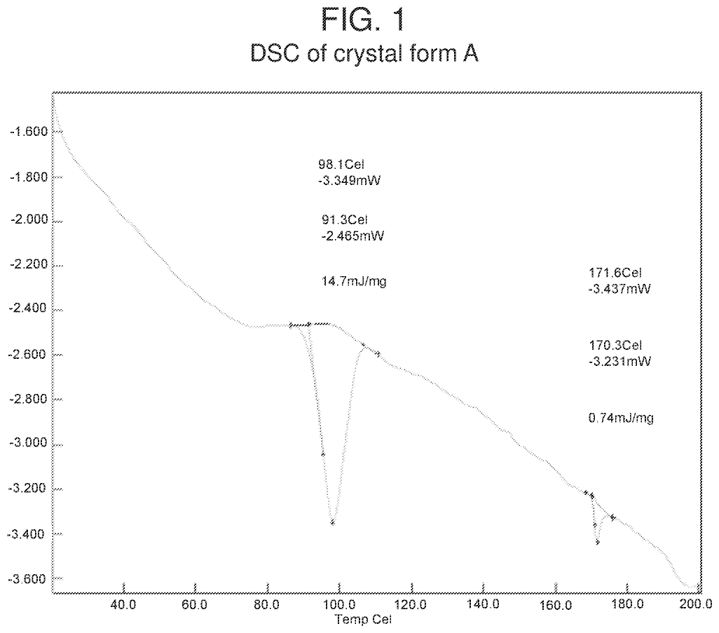

Crystal form A does not convert to an amorphous solid at temperatures below 65° C., however it does convert to amorphous solids at temperatures above 65° C., most preferably above 75° C. Differential scanning calorimetry (DSC) was performed on crystal form A of ajulemic acid. As seen in FIG. 1, DSC of crystal form A of ajulemic acid indicates an endothermic event with an onset of about 91° C. and a peak of about 98° C.

DSC Characterization of Crystal Form B

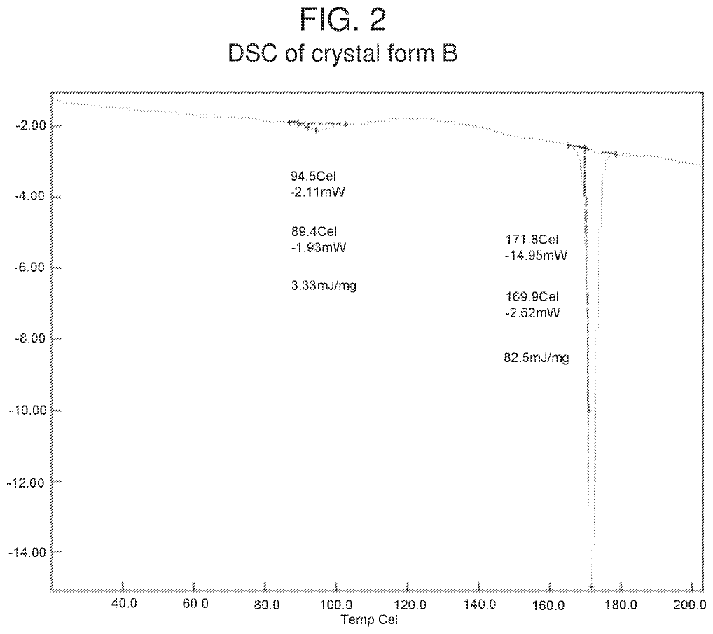

DSC analysis of the dried final isolated material showed a shallow endothermic event with onset at about 77° C. (corresponding to melting of trace amorphous material), followed by an exothermic event with onset at about 110° C. (corresponding to melted material recrystallizing as Form B). The main endothermic event, the melt of Form B, has an onset of about 170° C. and a peak at about 172° C. (FIG. 2).

### Example 8. X-Ray Powder Diffraction (XRPD) of Crystal Forms A and B

X-Ray Powder Diffraction (XRPD)

XRPD analysis was carried out on a PANalytical X'Pert Pro X-ray Diffractometer, scanning the samples between 3 and 35° 2θ. Material was loaded into a multi-well plate with mylar polymer film to support the sample. The multi-well plate was then placed into the diffractometer and analyzed using Cu K radiation (α₁ λ=1.54060 Å; α₂=1.54443 Å; β=1.39225 Å; α₁:α₂ ratio=0.5) running in transmission mode (step size 0.0130° 2θ) and using 40 kV/40 mA generator settings. Data was visualized and images generated using HighScore Plus 4.7 (PANalytical).

XRPD Characterization of Crystal Form A

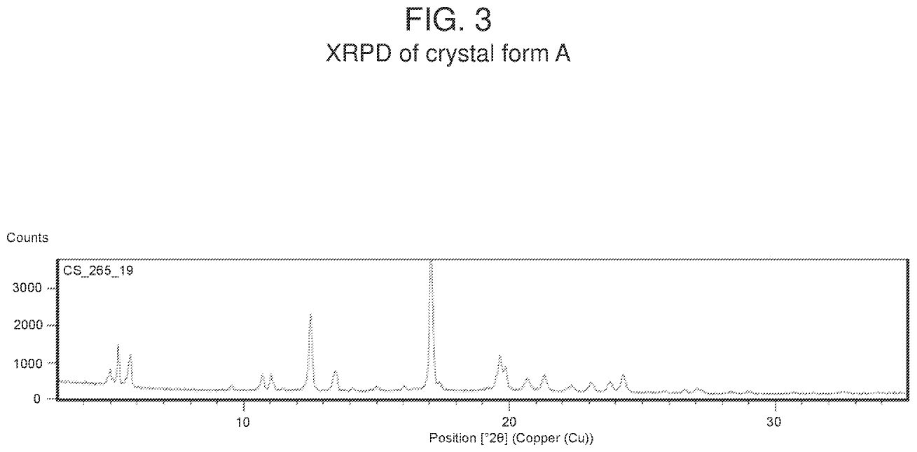

XRPD patterns of crystal form A were obtained. The XRPD diffraction angles 2θ (°) for crystal form A are provided in Table 5, below (showing all peaks with a relative intensity of equal to or greater than 10%). The XRPD trace of crystal form A is provided in FIG. 3.

TABLE 5
XRPD of Crystal Form A of Ajulemic Acid

| 2θ (°) | Relative Intensity (%) |
|---|---|
| 5.01 | 12 |
| 5.30 | 32 |
| 5.78 | 22 |
| 10.73 | 11 |
| 11.05 | 12 |
| 12.53 | 61 |
| 13.52 | 12 |
| 17.04 | 100 |
| 19.64 | 27 |
| 19.88 | 17 |
| 21.34 | 12 |
| 24.27 | 13 |

XRPD Characterization of Crystal Form B

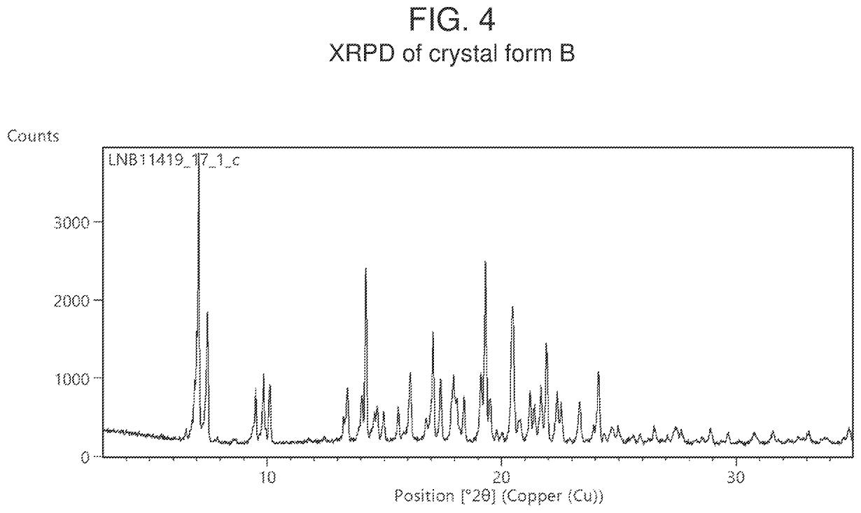

The XRPD also showed a unique pattern, distinct from either crystal form A or amorphous ajulemic acid. The XRPD trace for crystal form B is provided in FIG. 4 and the corresponding peaks are provided in Table 1, previously presented in the summary of the invention and replicated here for ease of reference (showing all peaks with a relative intensity of greater than or equal to 10%).

TABLE 1
(reproduced). XRPD of Crystal Form B of Ajulemic Acid

| 2θ (°) | Relative Intensity (%) |
|---|---|
| 7.09 | 100 |
| 7.47 | 82 |
| 9.53 | 26 |
| 9.85 | 33 |
| 10.12 | 25 |
| 13.40 | 21 |
| 14.00 | 14 |
| 14.22 | 56 |
| 14.56 | 13 |
| 14.70 | 21 |
| 14.96 | 14 |
| 16.09 | 33 |
| 17.05 | 54 |
| 17.37 | 23 |
| 17.93 | 23 |
| 18.06 | 21 |
| 18.39 | 17 |
| 19.08 | 34 |
| 19.27 | 80 |
| 19.50 | 19 |
| 19.78 | 11 |
| 20.38 | 31 |
| 20.46 | 45 |
| 21.19 | 23 |
| 21.38 | 13 |
| 21.63 | 12 |
| 21.87 | 48 |
| 22.31 | 19 |
| 22.51 | 11 |
| 23.28 | 23 |
| 24.05 | 18 |

Variable Temperature XRPD Characterization of Crystal Form B

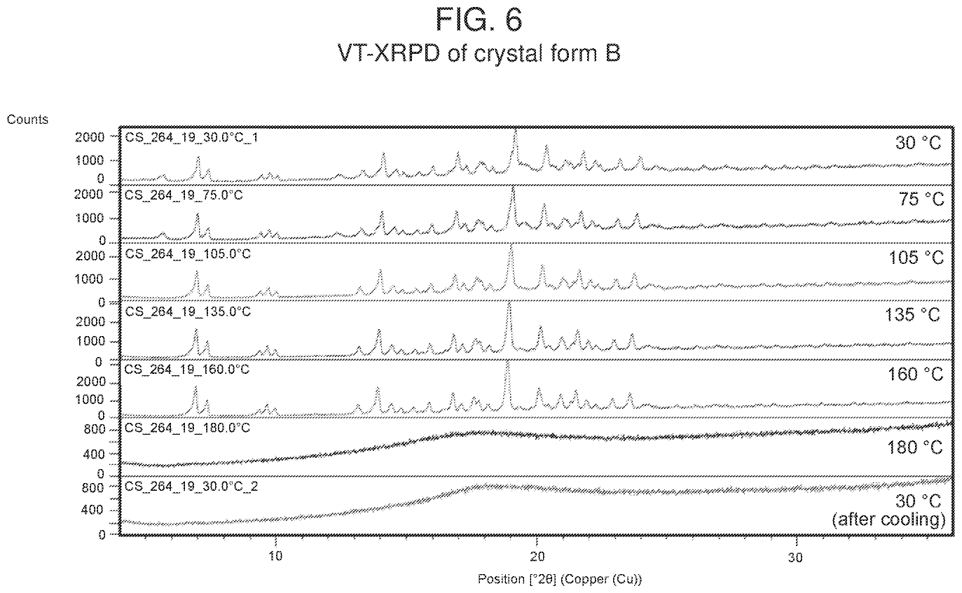

Variable temperature X-ray powder diffraction (VT-XRPD) was performed, with XRPD scans taken after a 5-minute hold at each temperature. The heating rate was 10° C./min, except from 135-160° C., where the heating rate was 1° C./min. The results of the VT-XRPD scan are provided in FIG. 6. VT-XRPD indicated that the endothermic event observed in DSC at approximately 170° C. is melt or decomposition of crystal form B. At temperatures above about 165-175° C., crystal form B of ajulemic acid may convert to an amorphous solid. The amorphous solid may be more susceptible to oxidative degradation than crystal form B and does not share the same XRPD or DSC signatures as either crystal form A or crystal form B.

Experimental vs Simulated XRPD of Crystal Form B

The XRPD of crystal form B was simulated using Software: CCDC Mercury 3.10.2; Build 189770. The Lorentz-polarisation correction assumes a laboratory X-ray source. No absorption is simulated. Fixed slit widths are assumed. No background is included. All non-hydrogen atoms are assumed to have isotropic atomic displacement parameters (Uᵢₛₒ) of 0.05 Å². Hydrogen atoms for which 3D coordinates are available are taken into account and assigned Uᵢₛₒ values of 0.06 Å². The powder pattern simulator takes site occupation factors into account. This corrects the patterns generated for disordered structures read from the CIF file. All reflections have a symmetric pseudo-Voight peak shape with a full width half maximum of 0.1° 2θ, corresponding to medium resolution laboratory data. The (0, 0, 0) reflection is excluded. The default °2θ resolution is 50.0 degrees, which, for the default CuKₐ1 radiation, corresponds to a direct space resolution of 3.0 Å. Experimental displacement parameters, either isotropic or anisotropic, are taken into account in the calculation.

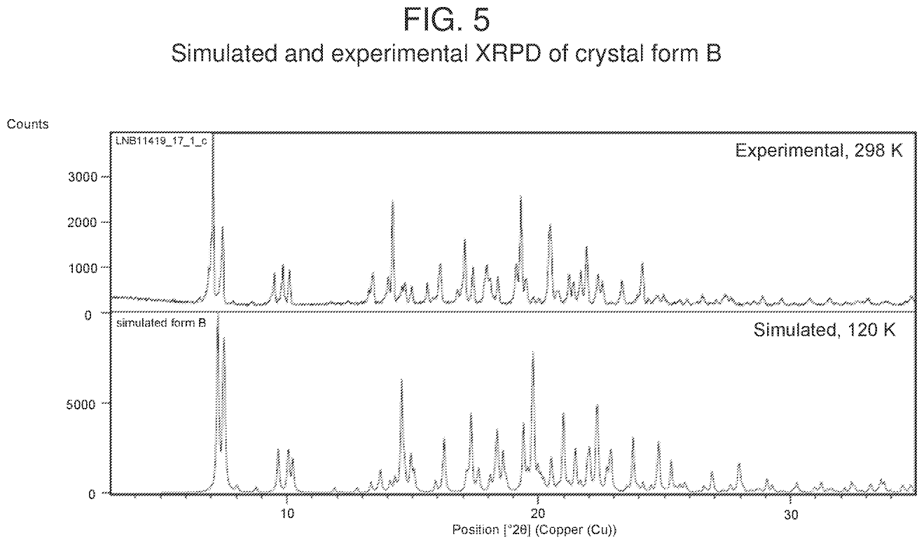

The simulated diffractogram (at 100 K) was compared to an experimental diffractogram (taken at 298 K) and they were found to be broadly consistent with one another (FIG. 5). Differences between the diffractograms are due to the different experimental temperatures.

### Example 9. Thermogravimetric Analysis/Dynamic Temperature Analysis (TGA/DTA) of Crystal Forms A and B

Thermogravimetric Analysis/Dynamic Temperature Analysis (TGA/DTA)

Approximately 5-10 mg of material was weighed into an open aluminum pan and loaded into a TA Instruments SDT650 and held at room temperature. The sample was then heated at a rate of 10° C./min from 30° C. to 350° C. during which time the change in sample weight was recorded along with the heat flow response (DSC). Nitrogen was used as the purge gas, at a flow rate of 100 cm³/min for both the sample and balance purges.

TGA/DTA Characterization of Crystal Form A

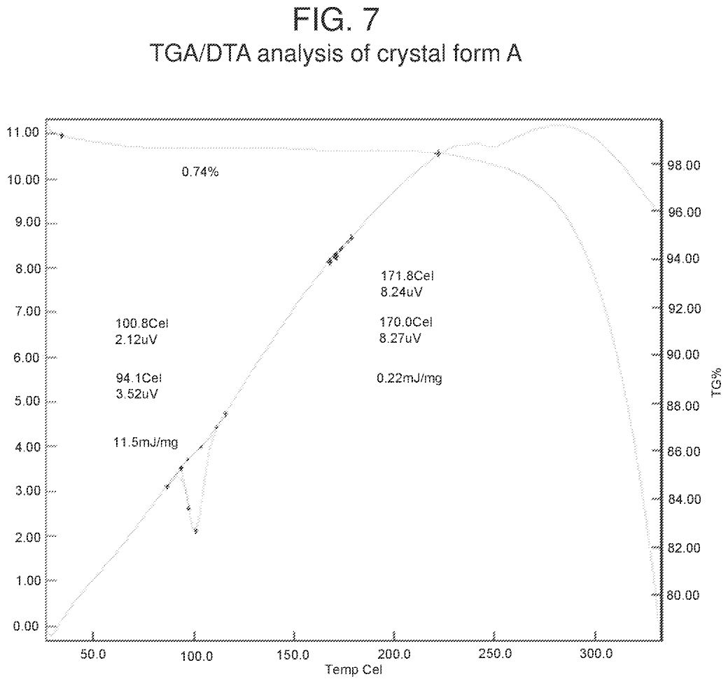

TGA/DTA indicates an endothermic event with an onset at about 94° C. TGA showed 0.7% wt. loss from the onset to about 210° C. (FIG. 7).

TGA/DTA Characterization of Crystal Form B

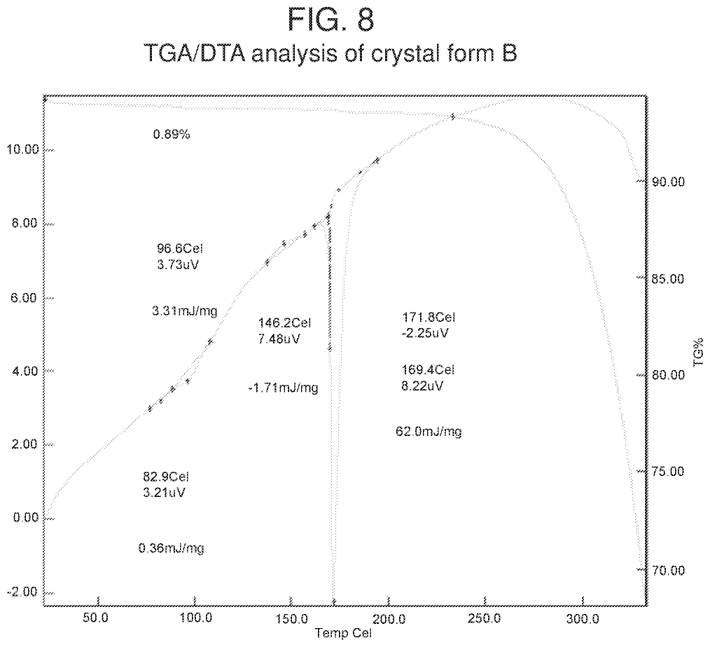

TGA/DTA indicates an endothermic event with an onset at about 169° C. TGA/DTA showed no significant mass loss until decomposition (at ca. 285° C.) (FIG. 8).

This TGA/DTA data demonstrates a higher melting point for crystal Form B compared to crystal Form A. The higher melting point may be advantageous to prevent the loss of crystallinity during manufacturing e.g., during the transient heating involved in tablet processing. The differences in the weight loss on heating between the crystal forms is likely ascribed to their different propensity for moisture sorption, as discussed in Example 10.

### Example 10. Dynamic Vapor Sorption (DVS) of Crystal Forms A and B

Approximately 10-20 mg of sample was placed into a mesh vapor sorption balance pan and loaded into a Surface Measurement Systems DVS Intrinsic dynamic vapor sorption balance. The material was subjected to a ramping profile from 40 to 90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH was carried out. Two cycles were performed. The weight change during the sorption/desorption cycles were plotted, allowing the hygroscopic nature of the sample to be determined. XRPD analysis was then carried out on any solid retained. DVS analysis indicated that crystal form B was slightly hygroscopic with circa 1 wt % water uptake at 90% RH. Post-DVS XRPD analysis showed no change in pattern.

By contrast, crystal Form A has been shown to uptake about 3.5% water by weight at 90% RH in similar DVS studies. Because this equilibration with ambient humidity is rapid, calculation or use of sufficiently accurate assay results on Form A material must take into account its current water content. Since the change experienced by crystal Form B material under identical conditions is significantly smaller, it is more likely to be negligible, which reduces the analytical burden imposed by use of this crystal form.

### Example 11. Nuclear Magnetic Resonance (NMR) of Crystal Form B

Nuclear Magnetic Resonance (NMR)

¹H and ¹³C experiments were performed on a Bruker AVIIIHD spectrometer (operating at 299.9 K, and 500.12 MHz for protons and 125.77 MHz for carbons). Experiments were performed in deuterated dimethyl sulfoxide and each sample was prepared to ca. 10 mM concentration.

NMR Characterization of Crystal Form B

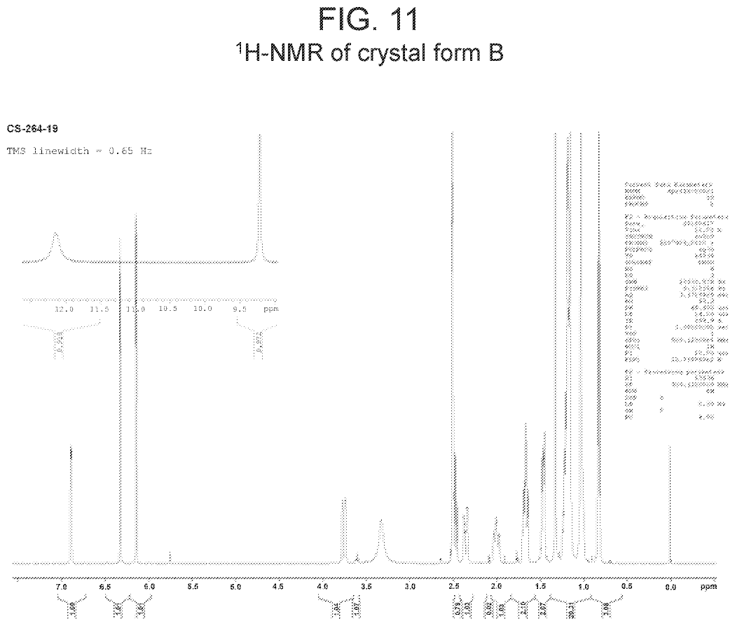

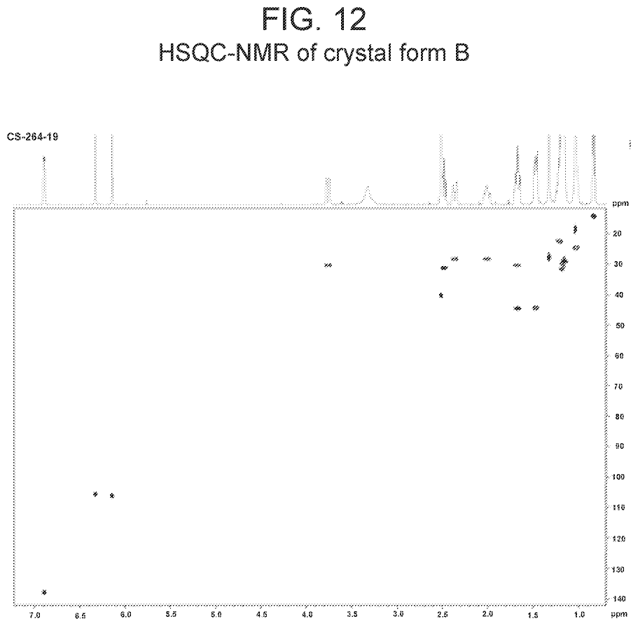

Crystal form B was characterized by proton (¹H) NMR (FIG. 11) and HSQC-NMR (FIG. 12). The NMR spectra are consistent with the structure of ajulemic acid.

### Example 12. Single Crystal X-Ray Diffraction Analysis (SCXRD) of Crystal Form B

Single crystal X-ray diffraction (SCXRD) analysis of a crystal form B was performed. A suitable crystal of ajulemic acid was selected and mounted in a loop using paratone oil. Data were collected using a Bruker D8Venture diffractometer equipped with a Photon III detector operating in shutterless mode at 100.0(2) K with Cu—Kα radiation (1.54178 Å). The structure was solved in the Olex2 software package (see Dolomanov, O. V., Bourhis, L. J., Gildea, R. J, Howard, J. A. K. & Puschmann, H. J. Appl. Cryst., 2009, 42, 339-341) with the SheIXT (intrinsic phasing) structure solution program (see Sheldrick, G. M. Acta Cryst., 2015, A71, 3-8) and refined with the SheIXL refinement package using Least Squares minimization (Sheldrick, G. M. Acta Cryst., 2015, C71, 3-8). Data were collected, solved and refined in the orthorhombic space-group P2₁2₁2₁.

All non-hydrogen atoms were located in the Fourier map and their positions refined prior to describing the thermal movement of all non-hydrogen atoms anisotropically. Within the asymmetric unit, two complete ajulemic acid formula units were refined. All hydrogen atoms were placed in calculated positions using a riding model with fixed Uiso at 1.2 times for all CH and CH₂ groups, and 1.5 times for all CH₃ and OH groups.

The highest residual Fourier peak was found to be 0.48 e.Å⁻³ approx. 1.18 Å from H(47A) and the deepest Fourier hole was found to be −0.26 e.Å⁻³ approx. 0.55 Å from C(48).

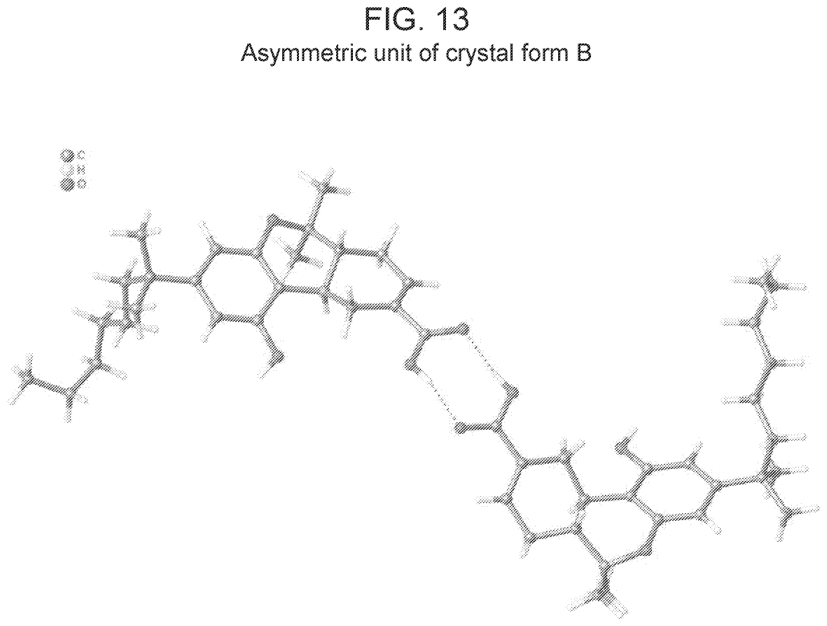

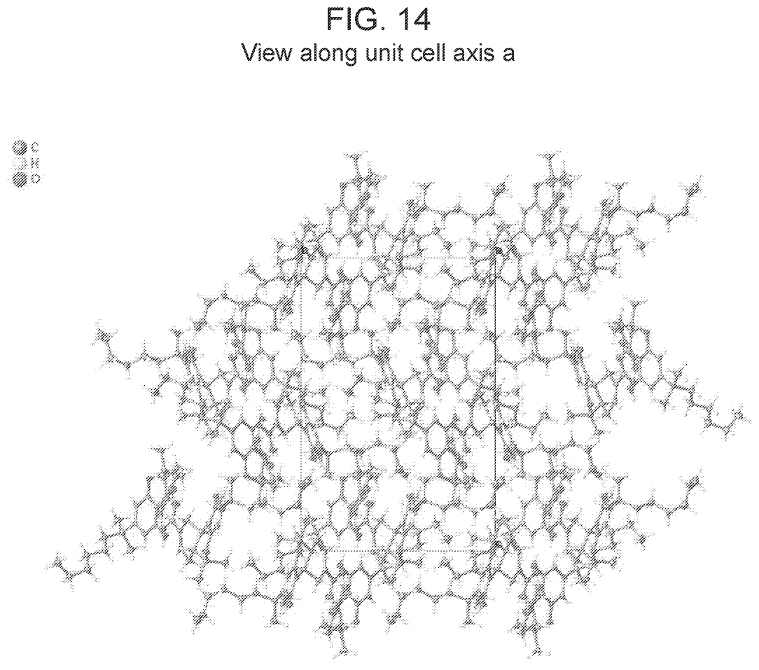

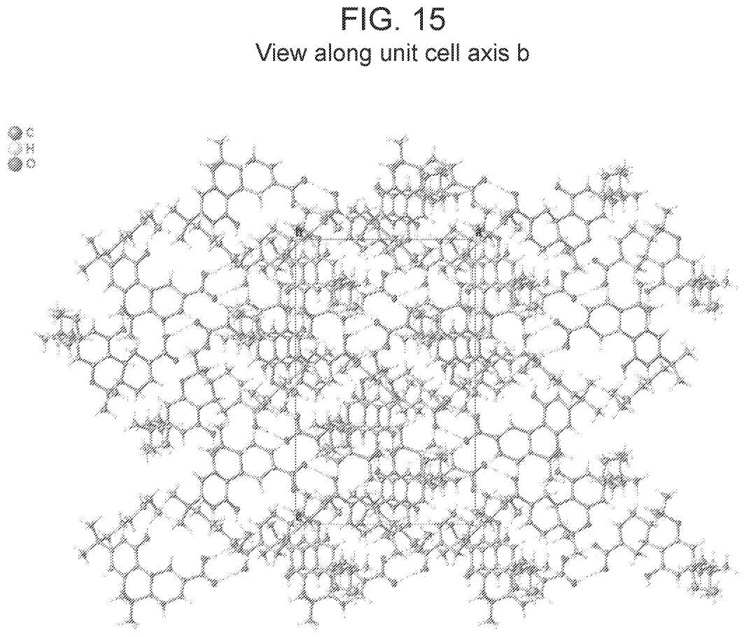

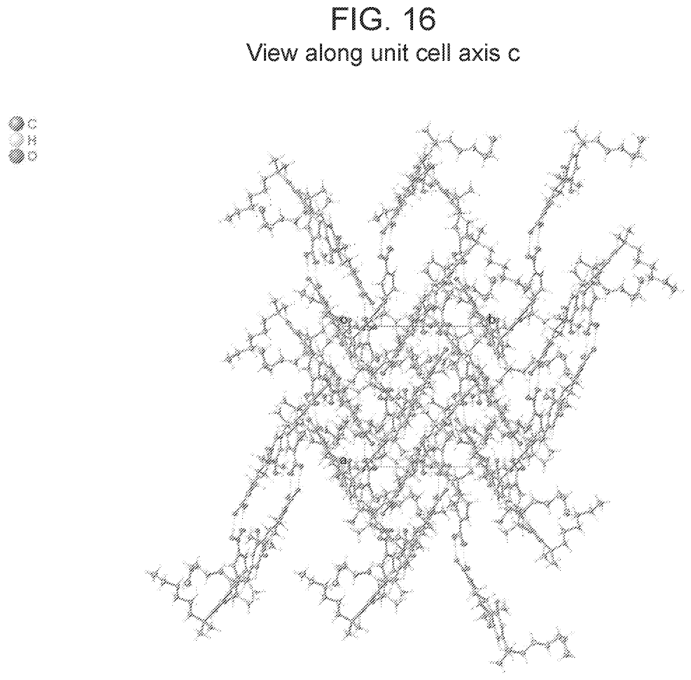

The asymmetric unit was found to contain two complete ajulemic acid formula units, with hydrogen bond association visible between the two molecules of ajulemic acid (FIG. 13). No solvent-accessible voids were found within the crystal structure when viewed along any of unit cell axes a, b, or c (FIGS. 14-16, respectively). The unit cell parameters and refinement indicators are provided below.

Unit Cell Parameters:
Crystal System: Orthorhombic
Space Group: P2₁2₁2₁
a=13.8951(3) Å
b=14.5553(3) Å
c=22.0051(4) Å
Z=8 Z′=2
α=90°, β=90°, γ=90°
Cell Volume: 4450.47(16) Å³
ρ=1.196 g/cm³

Refinement Quality Parameters
R₁(l>σ(l)=4.25%
wR₂ (all data)=11.15%
Flack x=0.06(7)
S=1.046
Rᵢₙₜ=9.10%

### Example 13. Thermodynamic Solubility Studies

For each experiment, approximately 50 mg of either crystal form A or crystal form B was weighed into a 20 mL scintillation vial. 15 mL of the appropriate solvent system was added and the solution was stirred at 37° C. At approximately 7.5 h, a portion of the mother liquor was transferred to a pre-heated vial (at 37° C.) via syringe filter (0.45 μm). An aliquot of slurry was removed, and the solids in the aliquot were isolated by centrifugation and plated for XRPD analysis. The plated solids were stored in freezer for 12 h prior to analysis.

The remaining solution was further stirred at 37° C. overnight (approximately 25 h total stirring time). Stirring was then terminated and the solids were allowed to settle. After approximately 0.75 h, a portion of the mother liquor was transferred to a pre-heated vial (at 37° C.) via syringe filter (0.45 μm). The residual solids were isolated by centrifugation and analyzed by XRPD.

For “dry” analysis, the XRPD plates were dried under vacuum at ambient temperature for approximately 23 h and re-analyzed. Each experiment was performed in triplicate, and the average results are provided in Table 6.

TABLE 6
Thermodynamic solubility studies

| | | After approximately | | | After approximately | | |
|---|---|---|---|---|---|---|---|
| | | 7.5 h at 37° C. | | | 25 h at 37° C. | | |
| | | Average | | | Average | | |
| pH | Input | Concentration | Form by XRPD | | Concentration | Form by XRPD | |
| media | Form | (μg/mL) | Wet | Dry | (μg/mL) | Wet | Dry |
| pH 6.1 | A | 3.0 | A | A | 2.0 | A + B | A + |
| | | | | | | | trace B |
| | B | 1.2 | B | B | 0.1 | B | B |
| pH 6.8 | A | 18 | A | A | 14 | A* | A* |
| | B | 4.3 | B | B | 2.0 | B | B |
| FaSSIF | A | 1063 | A | A | 391 | B + | B |
| | | | | | | trace A | |
| | B | 242 | B | B | 266 | (—) | B |
| FeSSIF | A | 335 | A* | A* | 227 | (X) + | (X) + |
| | | | | | | trace A | trace A |
| | B | 684 | B | B | 696 | B | B |
| FaSSGF | A | 0.2 | A + trace B | A + trace B | 0.2 | A + B | B + A |
| | B | 0.3 | B | B | 0.4 | B | B |

FaSSIF = Fasted State Simulated Intestinal Fluids;
FeSSIF = Fed State Simulated Intestinal Fluids;
FaSSGF = Fasted State Simulated Gastric Fluids;
*extra peaks observed;
(—) insufficient solids;
(X) unidentified diffractogram

### Example 14. Open Air Stability Study

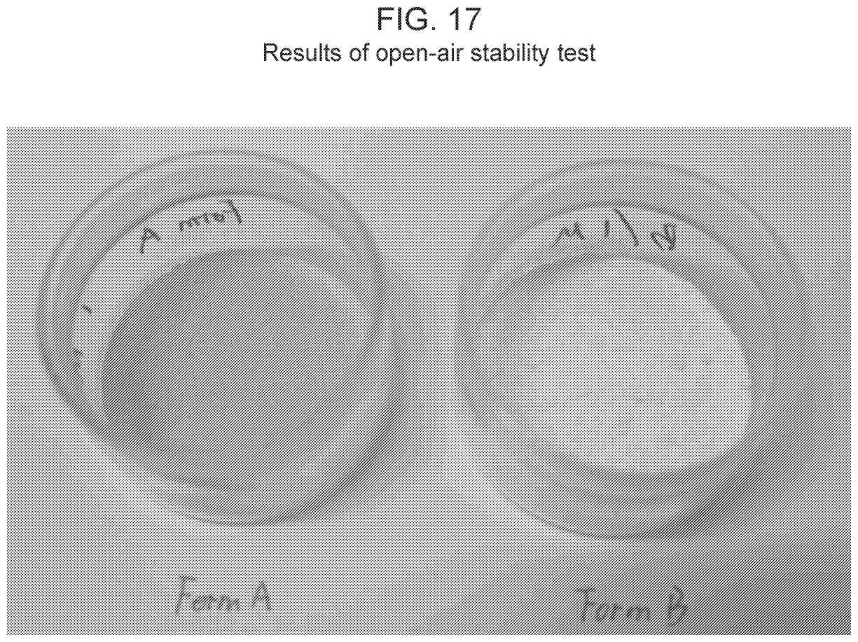

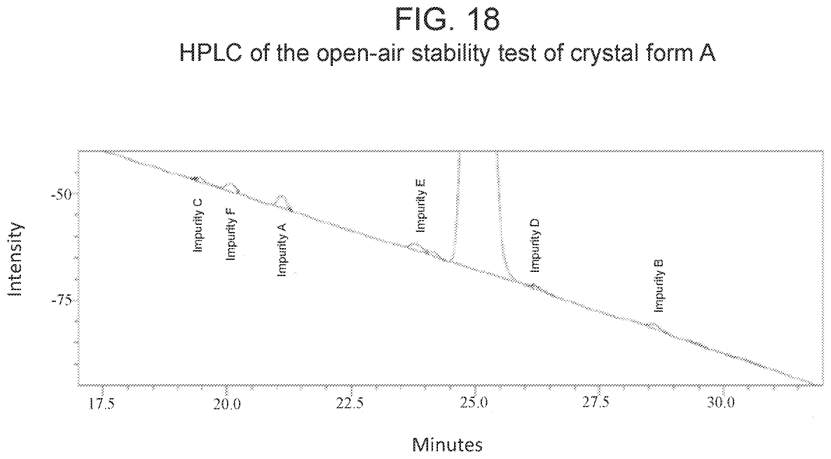

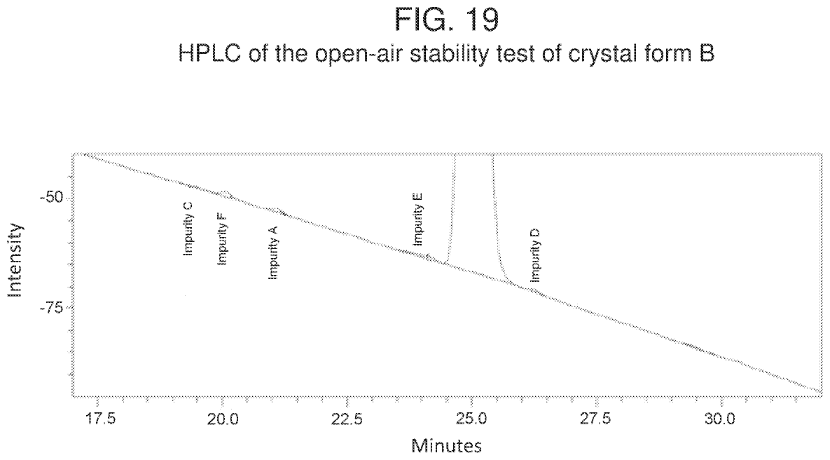

0.5 g of crystal forms A and B were stored in separate weighing bottles left open to atmosphere. The samples were stored for two months in a stability chamber at 40° C. and 75% relative humidity. The corresponding sample were analyzed for their visual appearance (Table 7), water content (Table 8), purity (Table 9), the presence of impurities Table 10) at the initial time point, at one month, and at two months. Impurity A, Impurity B, Impurity C, Impurity D, Impurity E, Impurity F correspond to impurities in the preparation. The water content, purity, and levels of impurities were determined by HPLC as described in the general methods. The data of Tables 7-10 demonstrate the increased stability of crystal form B to atmosphere. FIG. 17 is an image depicting crystal forms A and B after one month of the open air stability test demonstrating the greater stability of crystal form B. FIGS. 18 and 19 are HPLC chromatograms of crystal forms A and B after one month of the open air stability test.

TABLE 7
Appearance during open air stability study

| Form | Initial | 1 month | 2 months |
|---|---|---|---|
| Form A | Light orange | Orange powder | Dark Orange |
| | powder | | Powder |
| Form B | Light Yellow | Light yellow | Light Yellow |
| | Powder | powder | Powder |

TABLE 8
Water content during open air study

| Form | Initial | 1M | 2M |
|---|---|---|---|
| Form A | 2.38% | 2.10% | 2.03% |
| Form B | <0.05% | <0.05% | <0.05% |

TABLE 9
Purity of ajulemic acid (% area in HPLC) during open air study

| Form | Initial | 1M | 2M |
|---|---|---|---|
| Form A | 99.8% | 99.5% | 99.1% |
| Form B | 99.8% | 99.8% | 99.8% |

TABLE 10
Impurities observed (% area in HPLC) during open air study

| Form | Assay/Impurity | Initial | 1M | 2M |
|---|---|---|---|---|
| Form A | Ajulemic acid (% w/w) | 97.4% | 96.0% | 95.8% |
| | Impurity A | 0.07 | 0.20 | 0.37 |
| | Impurity B | <0.05 | <0.05 | 0.08 |
| | Impurity C | 0.08 | 0.09 | 0.11 |
| | Impurity D | <0.05 | <0.05 | <0.05 |
| | Impurity E | ND | 0.08 | 0.14 |
| | Impurity F | 0.12 | 0.12 | 0.12 |
| | Impurity RRT 0.96 | 0.08 | 0.09 | 0.08 |
| Form B | Ajulemic acid (% w/w) | 98.5% | 97.9% | 98.4% |
| | Impurity A | 0.05 | 0.05 | 0.06 |
| | Impurity B | ND | ND | ND |
| | Impurity C | <0.05% | <0.05 | <0.05 |
| | Impurity D | <0.05% | <0.05 | <0.05 |
| | Impurity E | <0.05% | <0.05 | <0.05 |
| | Impurity F | 0.07 | 0.07 | 0.07 |
| | Impurity RRT 0.96 | 0.05 | 0.05 | 0.05 |

0.05% = Limit of Quantification
ND = Not detected

### Example 15. Solid State Nuclear Magnetic Resonance (ssNMR) Characterization

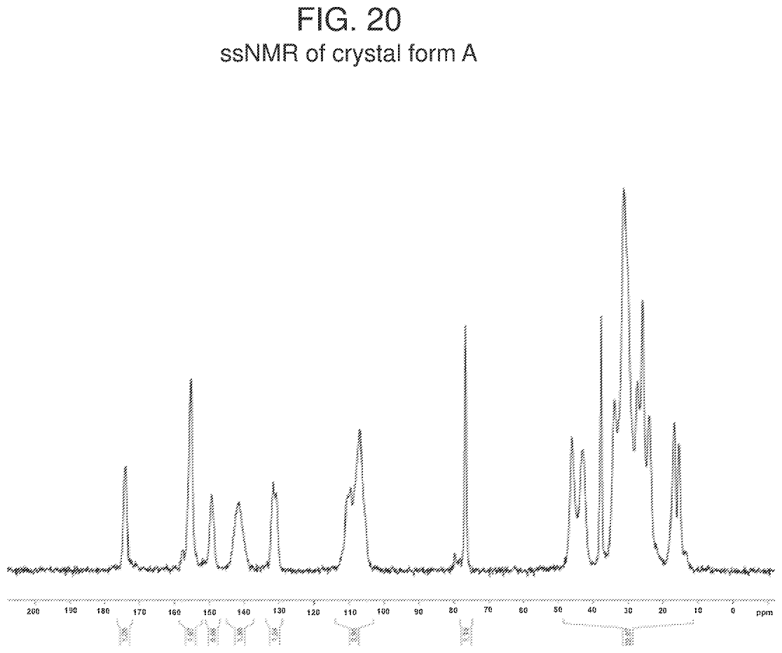

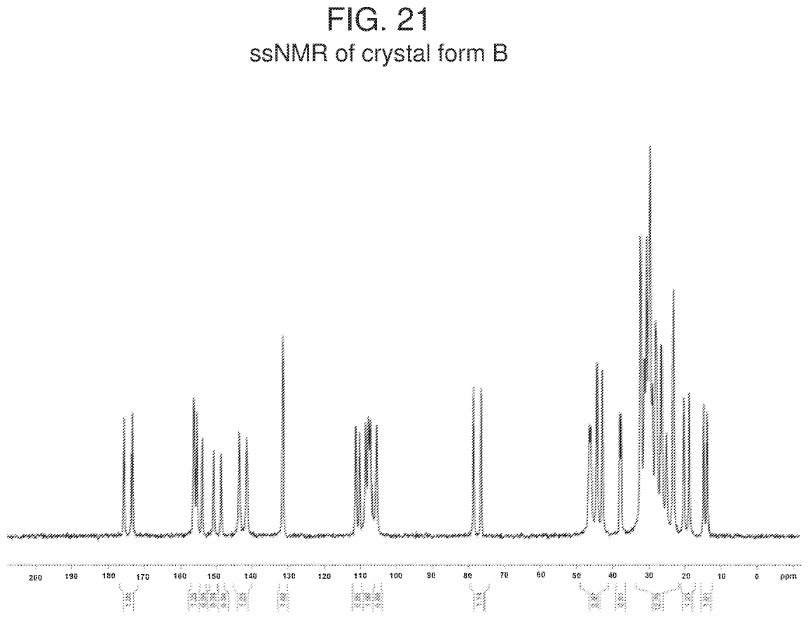

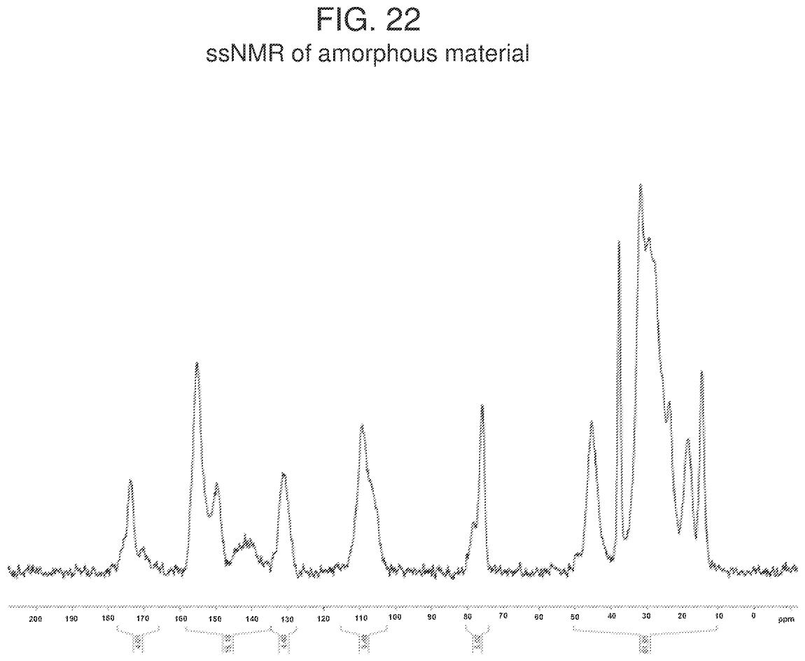

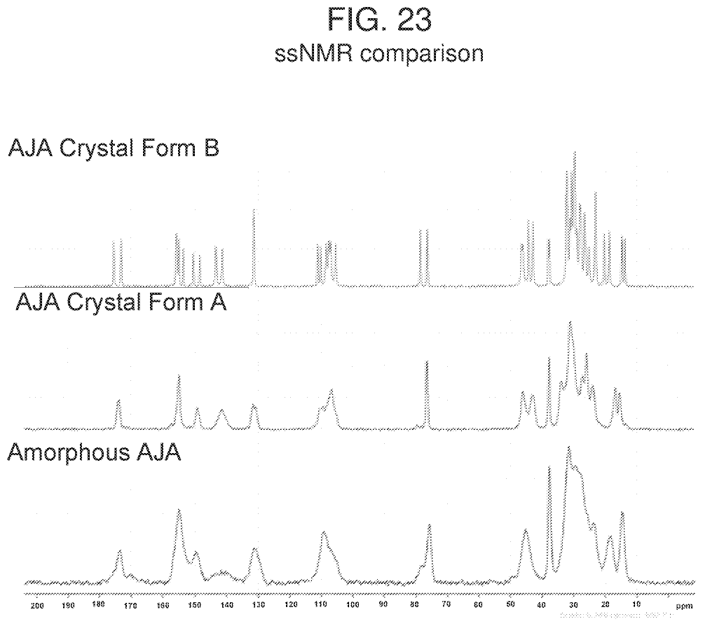

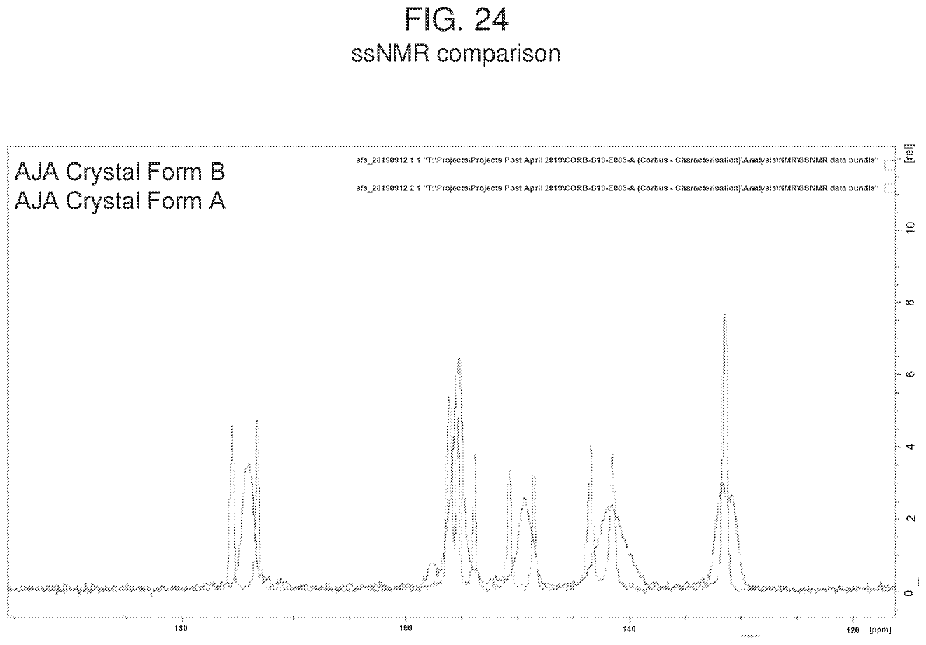

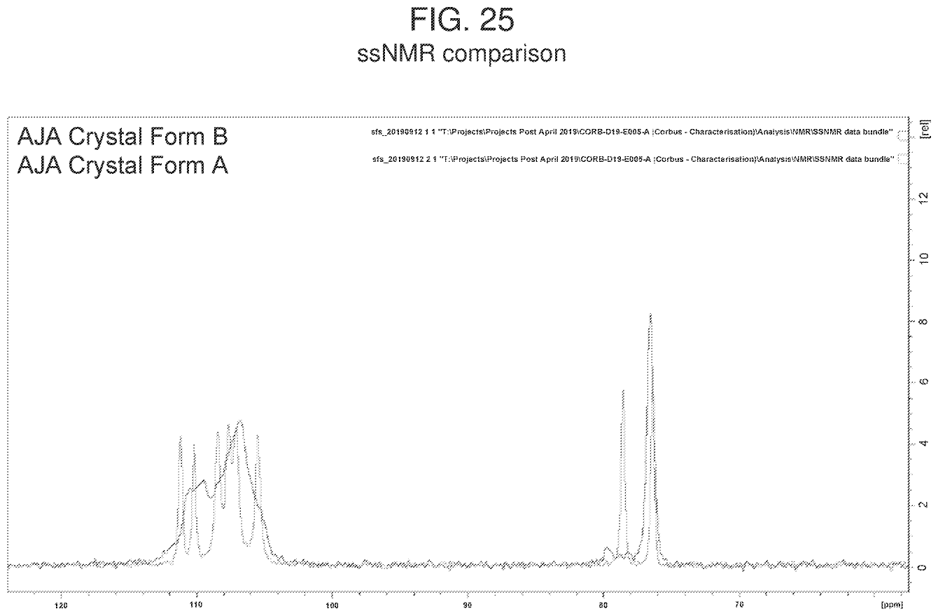

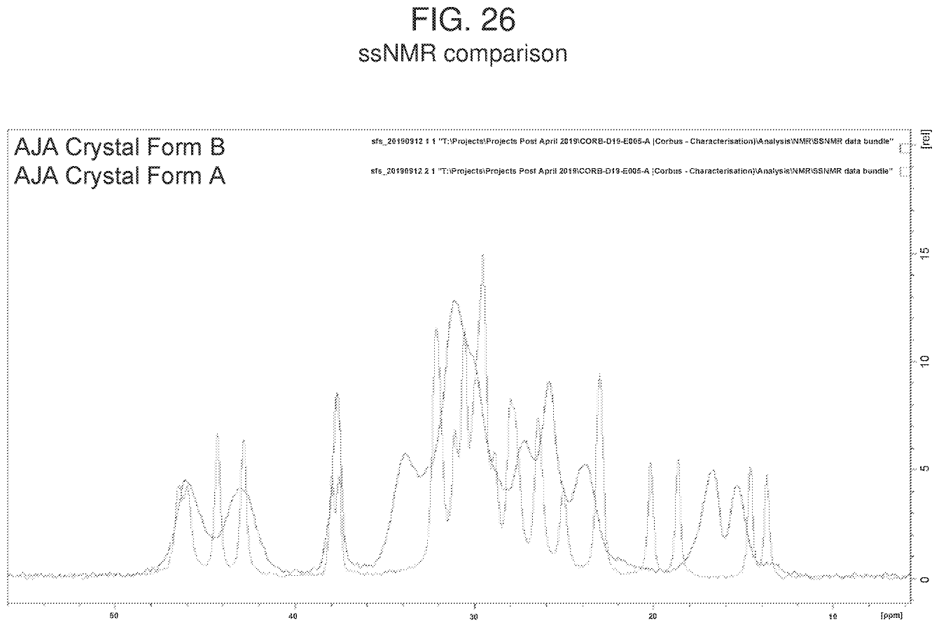

Crystal form A of ajulemic acid, crystal form B of ajulemic acid, and amorphous ajulemic acid were characterized by ¹³C ssNMR. ssNMR studies were performed on a Bruker Avance III HD spectrometer according to the experimental parameters provided in Table 11. The ssNMR spectrum for crystal form A is provided in FIG. 20, with the corresponding set of peaks provided in Table 12. The ssNMR spectrum for crystal form B is provided in FIG. 21, with the corresponding set of peaks provided in Table 2, previously presented in the summary of the invention and replicated here for ease of reference. The ssNMR spectrum for amorphous ajulemic acid is provided in FIG. 22, with the corresponding set of peaks provided in Table 13. Comparisons of the ssNMR spectra are provided in FIGS. 23-26.

TABLE 11
Experimental parameters for ¹³C ssNMR

| | Form A | Form B | Amorphous |
|---|---|---|---|
| Magnetic field | 9.4 T | | |
| Instrument name | Bruker Avance III HD spectrometer | | |
| (manufacturer/model) | | | |
| Spinning speed | 10 KHz | | |
| Rotor size | 4 mm (o.d.) | | |
| Decoupling field | 70 KHz | | |
| Contact time (CPMAS) | 4 ms | 4 ms | 4 ms |
| Number of scans | 3600 | 1800 | 1800 |
| Recycle delay | 1 s | 2 s | 1 s |

| | |
|---|---|
| Chemical shift reference | Neat tetramethylsilane |

TABLE 12
¹³C ssNMR of crystal form A of ajulemic acid
Peak v(F1) [ppm]

173.9
157.5
155.2
149.3
141.5
131.6
109.4
106.8
79.7
76.5
46.1
42.9
37.7
33.8
31.3
27.2
25.8
23.8
16.7
15.3

TABLE 2
(reproduced). ¹³C ssNMR of crystal form B of ajulemic acid
Peak v(F1) [ppm]

175.5
173.2
156.1
155.2
153.8
150.6
148.5
143.4
141.4
131.4
111.2
110.1
108.4
107.6
107.1
105.4
78.5
76.3
46.4
46.0
44.3
42.8
37.9
37.6
32.1
31.1
30.6
29.5
28.9
28.0
26.5
25.0
23.0
20.2
18.6
14.6
13.7

TABLE 13
¹³C ssNMR of amorphous ajulemic acid
Peak v(F1) [ppm]

173.6
155.2
149.8
141.9
131.4
109.2
75.7
45.2
37.6
31.6
29.1
23.5
18.2
14.6

### Example 16. Large-Scale Preparation of Crystal Form B

A solution of about 500 g of ajulemic acid dissolved in about 5 L of MTBE was concentrated under reduced pressure to a volume of 2 L. n-Heptane (2.5 L) was charged to the solution and the resulting mixture concentrated under reduced pressure to a volume of 2 L while maintaining the internal temperature below 40° C. The n-heptane addition/concentration sequence was repeated three more times. Analysis of the slurry showed that 0.01% MTBE remained. An additional 2.5 L of n-heptane were charged and the mixture warmed to 50° C. The slurry was stirred at this temperature for 14 h. A sample of the mixture was analyzed by XRPD and showed only Form B. The slurry was cooled to 25° C. and the solids isolated by vacuum filtration, washing the wet cake with 3 L of n-heptane. The cake was dried under vacuum at 40-50° C. to give 518 g of a yellow solid. The product was characterized by purity, assay, residual solvents, XRPD, and NMR, the results of which were consistent with the characterization of Form B described herein,

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. Crystals of ajulemic acid having at least one peak at diffraction angle 2θ at each of 7.1°±0.2°, 7.5°±0.2°, and 9.9°±0.2°, as measured by X-ray Powder Diffraction.

2. Crystals of ajulemic acid having at least one peak at diffraction angle 2θ at each of 7.1°±0.2°, 7.5°±0.2°, and 14.2°±0.2°, as measured by X-ray Powder Diffraction.

3. The crystals of claim 1 or 2, wherein the crystals have at least one peak at 143.4 ppm±0.2 ppm, as measured by ¹³C solid state Nuclear Magnetic Resonance.

4. The crystals of any one of claims 1-3, wherein the crystals have at least one peak at 150.6 ppm±0.2 ppm, as measured by ¹³C solid state Nuclear Magnetic Resonance.

5. The crystals of any one of claims 1-4, wherein the crystals have at least one peak at 153.8 ppm±0.2 ppm, as measured by ¹³C solid state Nuclear Magnetic Resonance.

6. Crystals of ajulemic acid having at least one peak at each of 143.4 ppm±0.2 ppm, 150.6 ppm±0.2 ppm, and 153.8 ppm±0.2 ppm, as measured by ¹³C solid state Nuclear Magnetic Resonance.

7. The crystals of ajulemic acid of claim 5 or 6, wherein the crystals have at least one peak diffraction angle 2θ of 7.1°±0.2° as measured by X-ray Powder Diffraction.

8. The crystals of ajulemic acid of any one of claims 5-7, wherein the crystals have at least one peak diffraction angle 2θ of 7.5°±0.2° as measured by X-ray Powder Diffraction.

9. The crystals of ajulemic acid of any one of claims 5-8, wherein the crystals have at least one peak diffraction angle 2θ of 14.2°±0.2° as measured by X-ray Powder Diffraction.

10. The crystals of ajulemic acid of any one of claims 5-9, wherein the crystals have at least one peak diffraction angle 2θ of 9.9°±0.2° as measured by X-ray Powder Diffraction.

11. Crystals of ajulemic acid having at least one peak at diffraction angle 2θ at each of 7.1°±0.2° and 7.5°±0.2°, as measured by X-ray Powder Diffraction, and at least one peak at each of 143.4 ppm±0.2 ppm and 150.6 ppm±0.2 ppm, as measured by ¹³C solid state Nuclear Magnetic Resonance.

12. Crystals of ajulemic acid having at least one peak at diffraction angle 2θ at each of 7.1°±0.2° and 14.2°±0.2°, as measured by X-ray Powder Diffraction, and at least one peak at each of 143.4 ppm±0.2 ppm and 150.6 ppm±0.2 ppm, as measured by ¹³C solid state Nuclear Magnetic Resonance.

13. Crystals of ajulemic acid having at least one peak at diffraction angle 2θ at each of 7.5°±0.2° and 14.2°±0.2°, as measured by X-ray Powder Diffraction, and at least one peak at each of 143.4 ppm±0.2 ppm and 150.6 ppm±0.2 ppm, as measured by ¹³C solid state Nuclear Magnetic Resonance.

14. The crystals of any one of claims 1-13, wherein the crystals have at least one peak at diffraction angle 2θ of 19.3°±0.2° as measured by X-ray Powder Diffraction.

15. The crystals of any one of claims 1-14, wherein the crystals have at least one peak at diffraction angle 2θ of 21.9°±0.2° as measured by X-ray Powder Diffraction.

16. The crystals of any one of claims 1-15, wherein the crystals have at least one peak at diffraction angle 2θ of 20.5°±0.2° as measured by X-ray Powder Diffraction.

17. The crystals of any one of claims 1-16, wherein the crystals have at least one peak at diffraction angle 2θ of 19.1°±0.2° as measured by X-ray Powder Diffraction.

18. The crystals of any one of claims 1-17, wherein the crystals have at least one peak at diffraction angle 2θ of 16.1°±0.2° as measured by X-ray Powder Diffraction.

19. The crystals of any one of claims 1-18, wherein the crystals have at least one peak at diffraction angle 2θ of 9.9°±0.2° as measured by X-ray Powder Diffraction.

20. The crystals of any one of claims 1-19, wherein the crystals have at least one peak at 175.5 ppm±0.2 ppm±0.2 ppm, as measured by ¹³C solid state Nuclear Magnetic Resonance.

21. The crystals of any one of claims 1-20, wherein the crystals have an endothermic onset at 170° C.±5° C. as determined by Differential Scanning Calorimetry.

22. The crystals of any one of claims 1-21, wherein the crystals have an endothermic peak at 172° C.±5° C. as determined by Differential Scanning Calorimetry.

23. A pharmaceutical composition comprising the crystals of any one of claims 1-22 and a pharmaceutically acceptable excipient.

24. The pharmaceutical composition of claim 23, wherein the pharmaceutical composition is a tablet.

25. The pharmaceutical composition of claim 23, wherein the pharmaceutical composition is a capsule.

26. A pharmaceutical composition comprising ajulemic acid, wherein the pharmaceutical composition is prepared by dissolving the crystals of ajulemic acid of any one of claims 1-23 into a suitable pharmaceutical excipient.

27. The pharmaceutical composition of claim 26, wherein the pharmaceutical excipient is selected from water, a saline solution, an oil, glycerol, an aqueous dextrose solution, propylene glycol, or ethanol.

28. The pharmaceutical composition of claim 27, wherein the oil is selected from petroleum oil, an animal oil, a vegetable oil, a mineral oil, or an oil of synthetic origin.

29. The pharmaceutical composition of any one of claims 26-28, wherein the pharmaceutical composition is a capsule.

30. The pharmaceutical composition of claim 29, wherein the capsule is a liquid capsule or a gel capsule.

31. The pharmaceutical composition of any one of claims 26-28, wherein the pharmaceutical composition is a liquid, and wherein the liquid is formulated for parenteral administration.

32. The pharmaceutical composition of claim 31, wherein the liquid is formulated for intravenous administration.

33. The pharmaceutical composition of any one of claims 26-28, wherein the pharmaceutical composition is a liquid, and wherein the liquid is formulated for ophthalmic administration.

34. The pharmaceutical composition of any one of claims 26-28, wherein the pharmaceutical composition is an ointment, and wherein the ointment is formulated for ophthalmic administration.

35. The pharmaceutical composition of any one of claims 26-28, wherein the pharmaceutical composition is a cream or an ointment, and wherein the cream or the ointment is formulated for topical administration.

36. The pharmaceutical composition of any one of claims 23-35, wherein the pharmaceutical composition is a unit dose comprising between 1 mg and 100 mg of ajulemic acid.

37. The pharmaceutical composition of claim 36, wherein the pharmaceutical composition is a unit dose comprising 5 mg±1 mg of ajulemic acid, 10 mg±2 mg of ajulemic acid, 20 mg±4 mg of ajulemic acid, or 40 mg±8 mg of ajulemic acid.

38. The pharmaceutical composition of any one of claims 23-37, wherein the pharmaceutical composition is administered once daily.

39. The pharmaceutical composition of any one of claims 23-37, wherein the pharmaceutical composition is administered twice daily.

40. A method of treating a subject having an inflammatory disease, the method comprising administering to the subject a pharmaceutical composition of any one of claims 23-39 in an amount sufficient to treat the inflammatory disease.

41. The method of claim 40, wherein the inflammatory disease is scleroderma, systemic lupus erythematosus, dermatomyositis, acquired immune deficiency syndrome (AIDS), multiple sclerosis, rheumatoid arthritis, psoriasis, diabetes, cancer, asthma, atopic dermatitis, an autoimmune thyroid disorder, ulcerative colitis, Crohn's disease, stroke, ischemia, a neurodegenerative disease, amyotrophic lateral sclerosis (ALS), chronic traumatic encephalopathy (CTE), chronic inflammatory demyelinating polyneuropathy, an autoimmune inner ear disease, uveitis, iritis, or peritonitis.

42. The method of claim 41, wherein the inflammatory disease is scleroderma.

43. The method of claim 42, wherein the scleroderma is selected from systemic sclerosis, localized scleroderma, or sine scleroderma.

44. The method of claim 41, wherein the inflammatory disease is systemic lupus erythematosus.

45. The method of claim 41, wherein the inflammatory disease is dermatomyositis.

46. The method of claim 41, wherein the diabetes is Type 1 diabetes.

47. The method of claim 41, wherein the neurodegenerative disease is Alzheimer's disease or Parkinson's disease.

48. A method of treating a subject having a fibrotic disease, the method comprising administering to the subject a pharmaceutical composition of any one of claims 23-39 in an amount sufficient to treat the fibrotic disease.

49. The method of claim 48, wherein the fibrotic disease is scleroderma, liver cirrhosis, interstitial pulmonary fibrosis, idiopathic pulmonary fibrosis, Dupuytren's contracture, keloids, cystic fibrosis, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, or organ fibrosis.

50. The method of claim 49, wherein the fibrotic disease is scleroderma.

51. The method of claim 50, wherein the scleroderma is selected from systemic sclerosis, localized scleroderma, or sine scleroderma.

52. The method of claim 49, wherein the fibrotic disease is cystic fibrosis.

53. The method of claim 49, wherein the organ fibrosis is dermal fibrosis, lung fibrosis, liver fibrosis, kidney fibrosis, or heart fibrosis.

54. A method of making a pharmaceutical composition comprising ajulemic acid, wherein the pharmaceutical composition is prepared by dissolving the crystals of ajulemic acid of any one of claims 1-22 into a suitable pharmaceutical excipient.

55. A method of producing the crystals of any one of claims 1-22, wherein ajulemic acid is dissolved in and subsequently isolated from heptanes.

56. A method of producing the crystals of any one of claims 1-22, wherein ajulemic acid is dissolved in and subsequently isolated from n-heptane.

57. A method of producing the crystals of any one of claims 1-22, wherein ajulemic acid is dissolved in and subsequently isolated from dichloromethane.

58. A method of producing the crystals of any one of claims 1-22, wherein ajulemic acid is dissolved in and subsequently isolated from water.

59. A method of producing the crystals of any one of claims 1-22, wherein ajulemic acid is dissolved in and subsequently isolated from cyclohexane.
